(19) United States
(12) Patent Application Publication
(10) Pub. No.: US 2014/0272949 A1
(43) Pub. Date: Sep. 18, 2014

(54) Methods for Fully Segregating Recombinant Marine Cyanobacteria

(71) Applicant: ALGENOL BIOFUELS INC., Bonita Springs, FL (US)

(72) Inventors: Ming-De Deng, Fort Myers, FL (US); Alexandra Friedrich, Berlin (DE); Kerstin Baier, Berlin (DE); Nien-Hsi Ko, Fremont, CA (US)

(73) Assignee: ALGENOL BIOFUELS INC., Bonita Springs, FL (US)

(21) Appl. No.: 13/833,102

(22) Filed: Mar. 15, 2013

(51) Int. Cl. C12P 7/06; C12N 15/74

(52) U.S. Cl. CPC C12P 7/065; C12N 15/74
USPC 435/6.11; 435/257.2

(57) ABSTRACT

Methods and compositions are provided for the full segregation of recombinant marine cyanobacteria.

### STATEMENT REGARDING FEDERALLY SPONSORED RESEARCH

This invention was made in part with United States government support under the Department of Energy grant number DE-EE0002867. The government has certain rights associated with this invention.

### REFERENCE TO SEQUENCE LISTING

This application contains a sequence listing submitted by EFS-Web, thereby satisfying the requirements of 37 C.F.R. §§1.821-1.825. The Sequence Listing, created on Mar. 15, 2013, is named “Sequence listing P0025.01.US_ST25”, and is 4 KB in size.

### BACKGROUND

The growing demand for energy and the environmental concerns about carbon dioxide emissions make the development of renewable biofuels more and more attractive. Biologically produced ethanol, which is one major type of biofuel, has been the focus of many academic and industrial efforts, see for example US published applications 2011/0151531 and 2011/0008861. There are different ways to generate ethanol through biological means. Current biological methods for producing ethanol from grains, seeds, and other cellulosic sources have been criticized for contributing to rising food prices and leading to deforestation or for lack of cost effectiveness. Biological production of ethanol from carbon dioxide and water using genetically modified microorganisms overcomes some of the problems associated with grain or cellulosic sources of ethanol and is a promising alternative. However, there is a need for improved methods of ethanol production in ethanologenic microorganisms to further facilitate implementation of this ethanol source.

In one example, recombinant cyanobacteria engineered to contain an exogenous ethanologenic expression cassette (e.g., a cassette expressing pyruvate decarboxylase (PDC) and an alcohol dehydrogenase (ADH)) can be used in the production of ethanol. The introduction of PDC and ADH into cyanobacteria enables light driven production of ethanol in these phototrophic bacteria by directing carbon fixed via photosynthesis into ethanol production. However, prolonged ethanol production by such recombinant cyanobacteria requires stable integration and maintenance of the ethanologenic expression cassette whether it is in a plasmid or in the chromosome.

### SUMMARY

This disclosure describes genetically modified marine cyanobacteria, methods of making such cyanobacteria, and methods for complete segregation of genetically modified marine cyanobacteria.

In an aspect, disclosed is a method for isolating recombinant marine cyanobacteria containing a recombinant form of a high copy number endogenous plasmid and lacking the wild-type form of the high copy number endogenous plasmid, the recombinant form of the high copy number endogenous plasmid is a wild-type high copy number endogenous plasmid backbone and an exogenous polynucleotide sequence insert, the exogenous polynucleotide sequence insert comprising a selective marker, the method contains the steps of a), culturing a recombinant marine cyanobacteria comprising the recombinant form of the high copy number endogenous plasmid on low salt media comprising concentrations of a selective agent, and b) analyzing plasmid DNA from colonies cultured on a medium of step a for the presence of said recombinant form of the high copy number endogenous plasmid and for the absence of the wild-type high copy number endogenous plasmid, and c) isolating recombinant marine cyanobacteria from step b that contain copies of said recombinant form of the high copy number endogenous plasmid and do not contain the wild-type high copy number endogenous plasmid.

In an aspect, disclosed is a method for isolating recombinant marine cyanobacteria containing a recombinant form of a high copy number endogenous plasmid and lacking the wild-type form of the high copy number endogenous plasmid, the recombinant form of a high copy number endogenous plasmid contains a wild-type high copy number endogenous plasmid backbone and an exogenous polynucleotide sequence insert, the exogenous polynucleotide sequence insert contains a selective marker, the method is as follows a) transforming marine cyanobacteria comprising the wild-type high copy number endogenous plasmid with the recombinant form of a high copy number endogenous plasmid, and b) culturing the transformed marine cyanobacteria in a high salt media having a selective agent, and c) culturing the cultured transformed marine cyanobacteria from step b on low salt media containing concentrations of a selective agent, and d) selecting individual colonies from a medium of step c, and e) culturing the selected individual colonies from step d on the medium of step d), and f) selecting colonies from the medium of step e, and g) culturing the selected colonies from step f on a high salt medium, and h) analyzing plasmid DNA from the high salt medium cultures from step g for the presence of the recombinant form of a high copy number endogenous plasmid and for the absence of the wild-type high copy number endogenous plasmid, and i) isolating recombinant marine cyanobacteria from step h that contain copies of the recombinant form of a high copy number endogenous plasmid and do not contain the wild-type endogenous plasmid. In an embodiment, the high copy number plasmid is present within the wild-type form of the marine cyanobacteria in a copy number per cell of greater than about 20, greater than about 25, greater than about 35, greater than about 45, greater than about 50, greater than about 60, or greater than about 70. In an embodiment, the wild-type endogenous plasmid is selected from the group consisting of pAQ1, and pAQ3. In an embodiment, the wild-type endogenous plasmid is pAQ1. In another embodiment, the low salt medium is a growth medium supplemented with less than about 15 g/L salt, less than 10 g/L salt or less than 5 g/L salt. In an embodiment, the high salt medium contains greater than about 35 g/L salt. In yet another embodiment, the selective agent is an antibiotic. In another embodiment, the selective marker is a polynucleotide sequence for an antibiotic resistance gene for an antibiotic. In yet another embodiment, the exogenous polynucleotide sequence is an expression cassette. In an embodiment, the expression cassette is an ethanologenic biosynthetic pathway expression cassette. In an embodiment, the ethanologenic biosynthetic pathway expression cassette has genes encoding pyruvate decarboxylase and alcohol dehydrogenase enzymes. In another embodiment, the analysis in step h is PCR analysis of DNA isolated from the isolated marine cyanobacteria from the high salt growth media. In an embodiment, the isolated recombinant marine cyanobacteria is selected from the group of Prochlorococcus, Synechocystis, Synechococcus, Chroococcales, Cyanobium, Oscillatoriales, Cyanobacterium, Chlorophyta, Pleurocapsales, Geitlerinema, Phormidium, Euhalothece, Anabaena, Lyngbya, Spirulina, Nostoc, Pleurocapsa, and Leptolyngbya. In an embodiment, the isolated recombinant marine cyanobacteria is of the species Synechococcus sp. strain PCC 7002. In another embodiment, the culturing of step c is on low salt agarose plates that have greater than about 1×, greater than about 5×, greater than about 10×, greater than about 20×, greater than about 30×, greater than about 40×, greater than about 50×, to greater than about 100× the minimum inhibitory concentration of the selective agent. In another embodiment, the culturing of step c is on low salt agarose plates having greater than about 15 μg/mL, 30 μg/mL, 60 μg/mL, 120 μg/mL, 240 μg/mL, 320 μg/mL, 400 μg/mL and 500 μg/mL of an antibiotic selected from the group consisting of streptomycin, spectinomycin, kanamycin, gentamycin, erythromycin, neomycin, rifampin, ampicillin, and zeomycin. In an embodiment, the culturing step of c is a parallel culturing on low salt agarose plates having greater than about 1×, greater than about 5×, greater than about 10×, greater than about 20×, greater than about 30×, greater than about 40×, greater than about 50×, to greater than about 100× the minimum inhibitory concentration of the selective agent. In yet another embodiment, the culturing step of c) is a stepwise culturing on low salt agarose plates first culturing on low salt agarose plates having greater than about 1×, then consecutive culturing on low salt agarose plates having greater than about 5×, greater than about 10×, greater than about 20×, greater than about 30×, greater than about 40× greater than about 50×, to greater than about 100× the minimum inhibitory concentration of the selective agent.

In an aspect, a method for isolating recombinant marine cyanobacteria containing a recombinant form of an endogenous plasmid and lacking the wild-type form of the endogenous plasmid is disclosed wherein the recombinant form of an endogenous plasmid comprising a wild-type endogenous plasmid backbone and an exogenous polynucleotide sequence insert, the exogenous polynucleotide sequence insert comprises a selective marker, the method includes: a) transforming marine cyanobacteria comprising the wild-type endogenous plasmid with the recombinant form of an endogenous plasmid, and b) culturing the transformed marine cyanobacteria in a high salt media having a selective agent, and c) culturing the cultured transformed marine cyanobacteria from step b on low salt media having concentrations of a selective agent, and d) selecting individual colonies from a medium of step c, and e) culturing the selected individual colonies from step d on the medium of step d), and f) selecting colonies from the medium of step e, and g) culturing the selected colonies from step f on a high salt medium, and h) analyzing plasmid DNA from the high salt medium cultures from step g for the presence of the recombinant form of an endogenous plasmid and for the absence of the wild-type endogenous plasmid, and i) isolating recombinant marine cyanobacteria from step h that contain copies of the recombinant form of an endogenous plasmid and do not contain the wild-type endogenous plasmid, and j) culturing the isolated recombinant marine cyanobacteria from step i in a high salt media that does not contain the selective marker for a period of time. In an embodiment, the time of step j is greater than about 1 day, 2 days, 1 week, 2 weeks, 3 weeks, 1 month, 2 months, 3 months, 4 months, 5 months, 6 months, 1 year, 2 years, 5 years or 10 years.

In another aspect, a fully segregated, genetically-modified marine cyanobacterial host cell is disclosed that has a recombinant form of a high copy number endogenous plasmid, wherein no corresponding endogenous plasmid is present in the host cell. In an embodiment, the host cell is selected from the group consisting of Prochlorococcus, Synechocystis, Synechococcus, Chroococcales, Cyanobium, Oscillatoriales, Cyanobacterium, Chlorophyta, Pleurocapsales, Geitlerinema, Phormidium, Euhalothece, Anabaena, Lyngbya, Spirulina, Nostoc, Pleurocapsa, and Leptolyngbya. In another embodiment, the host cell is Synechococcus sp. strain PCC 7002. In an embodiment, the host cell has a high copy number endogenous plasmid where the high copy number is greater than about 20, greater than about 25, greater than about 35, greater than about 45, greater than about 50, greater than about 60, or greater than about 70 copies per cell. In an embodiment, the endogenous plasmid is selected from pAQ1, and pAQ3. In another embodiment, the endogenous plasmid is pAQ1. In yet another embodiment, the high copy number plasmid contains a gene encoding pyruvate decarboxylase and a gene encoding alcohol dehydrogenase. In an embodiment, the host cell produces ethanol.

In an aspect, a fully segregated genetically modified Synechococcus sp. strain PCC 7002 host cell that has a recombinant form of a pAQ1-based endogenous plasmid and does not have the corresponding wild-type pAQ1 endogenous plasmid is present in the host cell. In an embodiment, the recombinant form of a pAQ1-based endogenous plasmid has a gene encoding pyruvate decarboxylase and a gene encoding alcohol dehydrogenase. In an embodiment, the host cell produces ethanol.

In an aspect, a method for isolating recombinant marine cyanobacteria containing a recombinant form of a high copy number endogenous plasmid and lacking the wild-type form of said high copy number endogenous plasmid is disclosed wherein the recombinant form of the high copy number endogenous plasmid contains a wild-type high copy number endogenous plasmid backbone and an exogenous polynucleotide sequence insert, and the exogenous polynucleotide sequence insert contains a selective marker under the control of the promoter of genes Tn903 or aadA, the method starts with culturing a recombinant marine cyanobacteria having the recombinant form of the high copy number endogenous plasmid on media containing about 18 g/L of salt and further containing various concentrations of a selective agent, and another step of analyzing plasmid DNA from colonies cultured on a medium of the first step for the presence of the recombinant form of the high copy number endogenous plasmid and for the absence of the wild-type high copy number endogenous plasmid, and also contains a step of isolating recombinant marine cyanobacteria from the media of the previous step that contain copies of the recombinant form of the high copy number endogenous plasmid and do not contain the wild-type high copy number endogenous plasmid.

In an aspect, A method for isolating recombinant marine cyanobacteria containing a recombinant form of a high copy number endogenous plasmid and lacking a wild-type form of said high copy number endogenous plasmid is disclosed, the recombinant form of the high copy number endogenous plasmid contains a portion of the wild-type form of the high copy number endogenous plasmid and an exogenous polynucleotide sequence insert, the exogenous polynucleotide sequence insert has a selective marker, a pyruvate decarboxylase gene, an alcohol dehydrogenase gene, and flanking regions wherein the flanking regions allow for homologous recombination of the exogenous polynucleotide sequence insert into the wild-type form of the high copy number endogenous plasmid to form the recombinant form of the high copy number endogenous plasmid, the method is as follows: a) transforming marine cyanobacteria having the wild-type high copy number endogenous plasmid with the exogenous polynucleotide sequence insert, and b) culturing the transformed marine cyanobacteria in a high salt medium having a selective agent, and c) culturing the cultured transformed marine cyanobacteria from step b on low salt media having concentrations of a selective agent, and d) selecting individual colonies of the transformed marine cyanobacteria from a medium of step c, and e) analyzing plasmid DNA from selected colonies of step d for the presence of the recombinant form of the high copy number endogenous plasmid and for the absence of the wild-type form of the high copy number endogenous plasmid, and f) isolating colonies from step e that contain the recombinant form of the high copy number endogenous plasmid and do not contain the wild-type form of the high copy number endogenous plasmid. In an embodiment, the recombinant marine cyanobacteria has a high copy number endogenous plasmid that is selected from the group consisting of pAQ1 and pAQ3. In another embodiment, the recombinant marine cyanobacteria is a host cell that produces ethanol.

In another aspect, a method for isolating recombinant marine cyanobacteria containing a recombinant form of a high copy number endogenous plasmid and lacking a wild-type form of the high copy number endogenous plasmid is disclosed where the recombinant form of the high copy number endogenous plasmid contains a portion of the wild-type form of the high copy number endogenous plasmid and an exogenous polynucleotide sequence insert, the exogenous polynucleotide sequence insert contains a selective marker, a pyruvate decarboxylase gene, and an alcohol dehydrogenase gene, the method contains the following steps: a) culturing a recombinant marine cyanobacteria having the recombinant form of the high copy number endogenous plasmid on low salt media having concentrations of a selective agent, and b) analyzing plasmid DNA from colonies cultured on a medium of step a for the presence of the recombinant form of the high copy number endogenous plasmid and for the absence of the wild-type high copy number endogenous plasmid, and c) isolating recombinant marine cyanobacteria from step b that contain copies of the recombinant form of the high copy number endogenous plasmid and do not contain the wild-type high copy number endogenous plasmid. In an embodiment, the recombinant marine cyanobacteria has a high copy number endogenous plasmid that is selected from the group consisting of pAQ1 and pAQ3. In another embodiment, the recombinant marine cyanobacteria is a host cell that produces ethanol.

In an aspect, a method for isolating recombinant marine cyanobacteria containing a recombinant form of a high copy number endogenous plasmid and lacking a wild-type form of the high copy number endogenous plasmid is disclosed, the recombinant form of the high copy number endogenous plasmid contains a portion of said wild-type form of the high copy number endogenous plasmid and an exogenous polynucleotide sequence insert, the exogenous polynucleotide sequence insert having a selective marker Tn903 or aadA, a pyruvate decarboxylase gene, and an alcohol dehydrogenase gene, the method is as follows: a) culturing recombinant marine cyanobacteria having the recombinant form of the high copy number endogenous plasmid on a media that has about 18 g/L of salt and concentrations of a selective agent, and b) selecting individual colonies of the transformed marine cyanobacteria from a medium of step a, and c) analyzing plasmid DNA from selected colonies of step b for the presence of said recombinant form of the high copy number endogenous plasmid and for the absence of said wild-type form of the high copy number endogenous plasmid, and d) isolating colonies from step c that contain the recombinant form of the high copy number endogenous plasmid and do not contain the wild-type form of the high copy number endogenous plasmid. In an embodiment, the recombinant marine cyanobacteria has a high copy number endogenous plasmid that is selected from the group consisting of pAQ1 and pAQ3. In another embodiment, the recombinant marine cyanobacteria is a host cell that produces ethanol.

In an aspect, a method for isolating recombinant marine cyanobacteria containing a recombinant form of a high copy number endogenous plasmid and lacking a wild-type form of the high copy number endogenous plasmid is disclosed, the recombinant form of the high copy number endogenous plasmid contains a portion of the wild-type form of the high copy number endogenous plasmid and an exogenous polynucleotide sequence insert, the exogenous polynucleotide sequence insert contains a selective marker that is Tn903 or aadA, a pyruvate decarboxylase gene, and an alcohol dehydrogenase gene, the method is as follows: a) culturing recombinant marine cyanobacteria having the recombinant form of the high copy number endogenous plasmid on a media having about 18 g/L of salt and concentrations of a selective agent, and b) selecting individual colonies of the transformed marine cyanobacteria from a medium of step a, and c) analyzing plasmid DNA from selected colonies of step b for the presence of the recombinant form of the high copy number endogenous plasmid and for the absence of the wild-type form of the high copy number endogenous plasmid, and d) isolating colonies from step c that contain the recombinant form of the high copy number endogenous plasmid and do not contain the wild-type form of the high copy number endogenous plasmid. In an embodiment, the recombinant marine cyanobacteria has a high copy number endogenous plasmid that is selected from the group consisting of pAQ1 and pAQ3. In another embodiment, the recombinant marine cyanobacteria is a host cell that produces ethanol.

The section headings used herein are for organizational purposes only and are not to be construed as limiting the described subject matter in any way. When definitions of terms in incorporated references appear to differ from the definitions provided in the present teachings, the definition provided in the present teachings shall control. It will be appreciated that there is an implied “about” prior to metrics such as temperatures, concentrations, and times discussed in the present teachings, such that slight and insubstantial deviations are within the scope of the present teachings herein. In this application, the use of the singular includes the plural unless specifically stated otherwise. Also, the use of “comprise,” “comprises,” “comprising,” “contain,” “contains,” “containing,” “include,” “includes,” and “including” are not intended to be limiting. It is to be understood that both the foregoing general description and the following detailed description are exemplary and explanatory only and are not restrictive of the invention. The articles “a” and “an” are used herein to refer to one or to more than one (i.e., to at least one) of the grammatical object of the article. By way of example, “an element” means one element or more than one element.

Unless otherwise defined, scientific and technical terms used in connection with the invention described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The nomenclatures utilized in connection with, and the laboratory procedures and techniques of described herein are those well-known and commonly used in the art, see, e.g., Sambrook et al., Molecular Cloning: A Laboratory Manual (Third ed., Cold Spring Harbor Laboratory Press, Cold Spring Harbor, N.Y. 2000).

As utilized in accordance with the embodiments provided herein, the following terms, unless otherwise indicated, shall be understood to have the following meanings:

The term “nucleic acid” and “nucleic acid molecule” refer to natural nucleic acid sequences such as DNA (deoxyribonucleic acid) and RNA (ribonucleic acid), artificial nucleic acids, analogs thereof, or combinations thereof.

As used herein, the terms “polynucleotide” and “oligonucleotide” are used interchangeably and mean single-stranded and double-stranded polymers of nucleotide monomers (nucleic acids), including, but not limited to, 2′-deoxyribonucleotides (nucleic acid) and ribonucleotides (RNA) linked by internucleotide phosphodiester bond linkages, e.g. 3′-5′ and 2′-5′, inverted linkages, for example, 5′-5′, branched structures, or analog nucleic acids. Polynucleotides have associated counter ions, such as H⁺, NH₄⁺, trialkylammonium, Mg²⁺, Na⁺ and the like. A polynucleotide can be composed entirely of deoxyribonucleotides, entirely of ribonucleotides, or chimeric mixtures thereof. Polynucleotides can be comprised of nucleobase and sugar analogs. Polynucleotides typically range in size from a few monomeric units, for example, 5-40 when they are more commonly referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytidine, “G” denotes deoxyguanosine, and “T” denotes thymidine

The term “plasmid” refers to a circular nucleic acid vector. Generally, plasmids contain an origin of replication that allows many copies of the plasmid to be produced in a bacterial (or sometimes eukaryotic) cell without integration of the plasmid into the host cell chromosome.

A “shuttle vector” refers to a vector which can propagate in two different host species.

An “integration vector” refers to a vector constructed to contain DNA fragments of an endogenous plasmid or chromosomal DNA flanking a nucleotide sequence of interest and can cause recombination at a homologous site on the endogenous plasmid or chromosomal DNA.

The term “construct” as used herein refers to a recombinant nucleic acid molecule that has been generated for the purpose of the expression of a specific nucleotide sequence or sequences, or is to be used in the construction of other recombinant nucleotide sequences. In general, “construct” is used herein to refer to a recombinant nucleic acid molecule.

An “expression cassette” or “cassette” refers to a set of polynucleotide sequences that permit transcription of a polynucleotide in a host cell. Expression cassettes may include, e.g., a promoter, a heterologous or native polynucleotide sequence that is transcribed, transcription termination signals, and enhancer elements.

An “expression vector” is a vector that permits the expression of a polynucleotide inside a cell. Expression of a polynucleotide includes transcriptional and/or post-transcriptional events. An expression vector may also refer to a vector that permits the expression of a polypeptide encoded by a polynucleotide on an expression vector.

An “expression construct” is an expression vector into which a nucleotide sequence of interest has been inserted in a manner so as to be positioned to be operably linked to the expression sequences present in the expression vector.

The term “host cell” as used herein refers to a cell into which DNA can be introduced by any appropriate means (e.g., transformation, transfection, electroporation, and conjugation).

The term “transformation” as used herein refers to a permanent or transient genetic change, e.g., a permanent genetic change induced in a cell following incorporation of non-host nucleic acid sequences.

The term “transformed cell” as used herein refers to a cell into which (or into an ancestor of which) an exogenous or heterologous nucleic acid molecule has been introduced, by means of recombinant nucleic acid techniques, a nucleic acid molecule encoding for a gene product of interest, for example, RNA and/or protein.

The term “culturing” signifies incubating a cell or organism under conditions wherein the cell or organism can carry out some, if not all, biological processes. For example, a cell that is cultured may be growing, reproducing, or capable of carrying out biological and/or biochemical processes such as replication, transcription, translation, etc.

The term “plating” as used herein refers to a method of culturing cells on solid growth media. For example, a volume of liquid medium containing cells is plated onto an agarose plate by spreading the liquid on the agarose plate.

The term “streaking” as used herein refers to the technique of isolating individual colonies on solid growth media through physically separating the individual colonies on a solid growth medium.

The term “homologous recombination” refers to the process of recombination between two nucleic acid molecules based on nucleic acid sequence similarity. The term embraces both reciprocal and nonreciprocal recombination (also referred to as gene conversion). A recombination event can be the result of equivalent or non-equivalent cross-over events. Equivalent crossing over occurs between two equivalent sequences or chromosome regions, whereas nonequivalent crossing over occurs between identical (or substantially identical) segments of nonequivalent sequences or chromosome regions. Unequal crossing over typically results in gene duplications and deletions. For a description of the enzymes and mechanisms involved in homologous recombination see, Watson et al., Molecular Biology of the Gene pp 313-327, The Benjamin/Cummings Publishing Co. 4th ed. (1987).

The term “expressed endogenously” refers to polynucleotides that are native, wild-type, to the host cell and are naturally expressed in the host cell.

The term “endogenous plasmid” refers to a plasmid present in the corresponding wild-type cyanobacterial strain.

The term “clone” refers to a group of substantially identical cells naturally derived from a common parent cell.

The term “recombinant form of an endogenous plasmid” refers to an endogenous plasmid that contains an exogenous polynucleotide sequence or sequences. A recombinant form of an endogenous plasmid may also contain a polynucleotide sequence or sequences derived from other endogenous plasmids or from chromosomal polynucleotides of the same cyanobacterial strain. A recombinant form of an endogenous plasmid differs from the corresponding wild-type endogenous plasmid in that it contains one or more exogenous polynucleotide sequences.

The term “high copy number” refers to a copy number of a plasmid that is greater than about 20 copies per cell. As an example, Synechococcus sp. strain PCC 7002 contains two species of high copy number endogenous plasmids, pAQ1 and pAQ3.

The term “backbone” refers to any portion of the DNA sequence of a vector or plasmid, e.g. endogenous plasmid, outside of any exogenous DNA of the plasmid or vector including the bacterial replicon region and sequences for plasmid/vector maintenance. In an example, a plasmid/vector backbone refers to the portion of a known plasmid that is a part of recombinant plasmids containing expression constructs, expression cassettes and/or other exogenous polynucleotide sequences integrated into the known plasmid.

The term “competent to express” refers to a host cell that provides a sufficient cellular environment for expression of endogenous and/or exogenous polynucleotides and/or polypeptides.

The term “selection marker” means a gene introduced into the cell that confers a trait suitable for artificial selection, the expression of which allows one to identify cells that have been transformed or transfected with a vector containing the marker gene. The selection marker may protect the organism from a selective agent (e.g., an antibiotic agent) that would normally kill it or prevent its growth. A selection marker may also confer a trait suitable through selection on media through counter-selection.

The term “antibiotic agent” refers to a drug or substance that kills (bactericidal) or inhibits the growth (bacteriostatic) of bacteria.

The term “hypersaline” refers to a solution having greater than about 50 parts per thousand (ppt) of dissolved salts.

The term “saline” generally refers to a solution having salt. In an embodiment, saline refers to a solution having from about 30 ppt to about 50 ppt of dissolved salts.

The term “brackish” refers to a solution having from about 0.5 ppt to about 30 ppt of dissolved salts.

The term “freshwater” refers to a solution having about 0 ppt to about 0.5 ppt of dissolved salts.

The term “marine cyanobacteria” refers to cyanobacteria capable of living in a saline solution. Marine cyanobacteria may also be capable of living in hypersaline, brackish, and freshwater solutions.

The term “high salt” generally refers to solutions having greater than about 35 ppt dissolved salts.

The term “low salt” generally refers to solutions having less than about 15 ppt dissolved salts.

The terms “high salt” and “low salt” may refer to relative amounts of dissolved salts when used for comparative purposes.

The term “low light” is a relative term and is dependent upon the cyanobacterium strain, and culturing conditions. In one example, low light is defined as about less than 300 μEs⁻¹ m⁻² on the surface of a culture.

The term “high light” is a relative term and is dependent upon the cyanobacterium strain, and culturing conditions. In one example, high light is defined as about more than 2000 μEs⁻¹ m⁻² on the surface of a culture.

The term “deg”, as used in gene names, denotes degenerated versions of the corresponding wild type genes.

Terms designating promoter sequences, which control the transcription of a certain gene in their native form are given by a capitalized letter “P” followed by the gene name. For example “PziaA” is the promoter controlling the transcription of the ziaA gene.

The following figures, description, and examples illustrate certain embodiments of the present disclosure in detail. Those of skill in the art will recognize that there are numerous variations and modifications that are encompassed by its scope. Accordingly, the description of certain embodiments should not be deemed to limit the scope of the present disclosure.

### BRIEF DESCRIPTION OF DRAWINGS

FIG. 1 depicts a plasmid map of pAB104.

FIG. 2 depicts a plasmid map of pAB114.

FIG. 3 depicts a polynucleotide sequence of an integration vector flanking region, Flank A3.

FIG. 4 depicts a polynucleotide sequence of an integration vector flanking region, Flank B3.

FIG. 5 depicts a plasmid map of pAB125.

FIG. 6 depicts a plasmid map of pAQ1 showing the location of the various flanking regions described herein.

FIG. 7 is an image of an ethidium bromide-stained agarose gel depicting PCR analysis of DNA from high-salt medium unsuccessfully segregated Synechococcus sp. strain PCC 7002 transformed with pAQ1-based shuttle vectors pAB220, pAB224 and pAB225.

FIG. 8 is an image of an ethidium bromide-stained agarose gel depicting PCR analysis of pAB125 obtained from transformed, low-salt medium segregated Synechococcus sp. strain PCC 7002.

FIG. 9 is a bar graph depicting levels of ethanol production in fully segregated clones and non-segregated control clones of Synechococcus sp. strain PCC 7002. transformed with integrative vector pAB125 (pAQ1B3A3:Pnb1A-PDC-Prbc-ADH_{deg}).

FIG. 10 is an image of an ethidium bromide-stained agarose gel depicting PCR analysis of recombinant and WT plasmids in both segregated clones (numbers 4, 11, 18, and 19) and non-segregated clones, numbers 1, and 7) of Synechococcus sp. strain PCC 7002 transformed cells.

FIG. 11 is an image of an ethidium bromide-stained agarose gel depicting PCR analysis of pAB232 obtained from transformed, low-salt medium segregated Synechococcus sp. strain PCC 7002.

FIG. 12 depicts a plasmid map of TK161.

FIG. 13 depicts a plasmid map of TK162.

FIG. 14 is an image of an ethidium bromide-stained agarose gel depicting PCR analysis of recombinant pAQ3 (containing the integrative fragment of TK161) and wild-type plasmids in segregated clones of Synechococcus sp. strain PCC7002 (lanes 1-8) and of wild type pAQ3 (lane 9).

FIG. 15 is an image of an ethidium bromide-stained agarose gel depicting PCR analysis of recombinant pAQ3 (containing the integrative fragment of TK162) and wild-type plasmids in both segregated clones, non-segregated clones (lanes 4, 7), and of wild type pAQ3 (lane 21).

FIG. 16 depicts ethanol production by fully segregated Synechococcus sp. strain PCC 7002 cells transformed with TK161.

FIG. 17 depicts ethanol production by fully segregated Synechococcus sp. strain PCC 7002 cells transformed with TK162.

FIG. 18 depicts a polynucleotide sequence of an integration vector flanking region, pAQ1-FA2.

FIG. 19 depicts a polynucleotide sequence of an integration vector flanking region, pAQ1-FB2.

FIG. 20 depicts a plasmid map of TK165.

FIG. 21 depicts a plasmid map of TK166.

FIG. 22 is an image of an ethidium bromide-stained agarose gel depicting PCR analysis of recombinant pAQ1 (containing the integrative fragment of TK165) [lanes 1,2] in fully segregated clones of Synechococcus sp. strain PCC 7002 transformed cells. Lane 3 depicts the PCR product from the wild-type pAQ1 plasmid.

FIG. 23 is an image of an ethidium bromide-stained agarose gel depicting PCR analysis of recombinant pAQ1 (containing the integrative fragment of TK166) and wild-type pAQ1 plasmids in fully segregated (lane 10), partially segregated (e.g. lanes 7,9,11. 16) and non-segregated clones (lanes 1-5) of Synechococcus sp. strain PCC 7002 transformed cells. Lane 17 depicts the PCR product from the wild-type pAQ1 plasmid.

FIG. 24 depicts ethanol production by fully segregated Synechococcus sp. strain PCC 7002 cells transformed with TK165.

FIG. 25 depicts ethanol production by fully segregated Synechococcus sp. strain PCC 7002 cells transformed with TK166.

### DETAILED DESCRIPTION

Disclosed herein are methods for obtaining full segregation of marine cyanobacteria containing recombinant forms of endogenous plasmids in high copy numbers.

The methods and vectors described herein can be used to produce higher amounts of ethanol and/or other products in cyanobacterial cells including marine cyanobacteria and cyanobacteria capable of growing in hypersaline conditions. Recombinant genes of interest are placed into an extrachromosomal plasmid that is naturally present in the cyanobacterium strain, an endogenous plasmid.

Without being bound by theory, it is believed that a given endogenous plasmid in a given cyanobacterium host has to carry one or more genes that are essential to the host cell either permanently or under certain conditions. Otherwise, natural selection would have eliminated the plasmid. As compared to the use of heterologous plasmids, plasmids derived from an endogenous plasmid (or recombinant form of an endogenous plasmid) could be more stable when they have been fully segregated, i.e., all the wild type endogenous plasmid copies have been replaced with its recombinant form. The fully segregated recombinant form of an endogenous plasmid is likely to remain in the cell after many generations. The fully segregated recombinant form of an endogenous plasmid remains even under negative selection pressure resulting from the expression of the gene or genes incorporated into the recombinant plasmid. It remains even when no antibiotic or other selection agent is used to select for the recombinant plasmid.

Therefore, it is possible to use cyanobacteria strains containing fully segregated recombinant forms of an endogenous plasmid carrying gene(s) of interest in large-scale production without the need for the use of antibiotics or other selection agents. Because the recombinant form of an endogenous plasmid is genetically stable, there is an increase in the length of time that the desired product can be successfully produced in a culture. This is useful, for example, in situations where large scale recombinant cyanobacterial cultures are grown for several days, weeks, months or years at a time to produce a commercial product.

Some cyanobacteria contain plasmids at higher copy numbers than their chromosome. Therefore, genes integrated into the high copy number plasmid could be expressed at higher levels than if it were chromosomally integrated.

Disclosed herein are methods for obtaining marine cyanobacteria containing recombinant forms of endogenous plasmids which are maintained within the marine cyanobacteria for a period of at least two weeks to about five months or longer.

In an embodiment, methods are disclosed for obtaining fully segregated marine cyanobacteria populations. The segregated marine cyanobacteria contain recombinant forms of endogenous plasmids but do not contain the corresponding wild-type endogenous plasmid.

In an embodiment, the segregation of marine cyanobacteria containing the recombinant form of an endogenous plasmid from those containing wild-type endogenous plasmid takes place in freshwater or brackish conditions containing a selective agent. The fully segregated marine cyanobacteria are then cultured and/or grown under saline and/or hypersaline conditions that do not contain the selective agent.

### Cyanobacteria

Cyanobacteria, also known as blue-green algae, are photosynthetic bacteria widespread in marine and freshwater environments. Cyanobacteria have simple growth requirements; grow to high densities, and use light, carbon dioxide, and other inorganic nutrients efficiently. They have been identified as attractive hosts for production of valuable organic products. Because of these features, cyanobacteria are highly suitable for the production of ethanol or other products by the bioconversion of solar energy and carbon dioxide.

Marine cyanobacteria are adapted to life in seawater or other solutions containing salt at about 3.5 weight percent, 35 g/L, and 35 ppt. Synechococcus sp. strain PCC 7002 is a marine cyanobacterium that preferably grows in brackish and/or saline water but can also grow in freshwater solutions or media. The organism is unicellular or forms short filaments of two to four cells at temperatures near the optimal temperature for growth of 38° C. The cells are 1.5-2.5 μm in size and lack phycoerythrin or phycoerythrocyanin. Synechococcus sp. strain PCC7002 is facultatively photoheterotrophic with glycerol as substrate, has an obligate requirement for vitamin B₁₂, and is naturally transformable. It is among the fastest growing of all cyanobacteria, with a doubling time under optimal conditions of about 3.5 hours. Optimal conditions for the growth of Synechococcus sp. strain PCC 7002 are conditions wherein light intensity is equal to about 250 μE m⁻² s⁻¹, the nitrogen source is urea, and CO₂ content is about 2% (v/v). The strain is tolerant of high light intensities and has been grown at light intensities as high as 5000 μE m⁻² s⁻¹.

Other marine cyanobacterial species include, but are not limited to, Prochlorococcus, Synechocystis, various other Synechococcus, Chroococcales, Cyanobium, Oscillatoriales, Cyanobacterium, Chlorophyta, Pleurocapsales, Geitlerinema, Phormidium, Euhalothece, Anabaena, Lyngbya, Spirulina, Nostoc, Pleurocapsa, and Leptolyngbya.

Some marine and other cyanobacteria can grow under hypersaline conditions. Exemplary genera containing at least some hypersaline species include but are not limited to Nostocales, Leptolyngbya, Cyanothece, and Euhalothece.

Freshwater cyanobacteria are capable of growing in waters containing about 0.5 ppt dissolved salts are less. Freshwater cyanobacteria are generally propagated on a low salt medium well known to those of skill in the art (e.g., BG-11).

For optimal growth, some marine cyanobacteria are propagated on a low salt media supplemented with additional salt (i.e., media is supplemented with NaCl, natural sea water, or artificial seawater (such as Instant Ocean, for example, available from Spectrum Brands Company) to achieve the desired salt content). Cultures can be maintained at 28° C. to 37° C. and bubbled continuously with 5% CO₂.

In an embodiment, Synechococcus sp. strain PCC 7002 cells may be cultured in BG-11 media, available for purchase from Sigma-Aldrich, Saint Louis Mo. BG-11 media or “MBG-11” contains about 17.65 mM NaNO₃, 0.18 mM K₂HPO₄, 0.3 mM MgSO₄, 0.25 mM CaCl₂, 0.03 mM citric acid, 0.03 mM ferric ammonium citrate, 0.003 mM EDTA, 0.19 mM Na₂CO₃, 2.86 mg/L H₃BO₃, 1.81 mg/L MnCl₂, 0.222 mg/L ZnSO₄, 0.390 mg/L Na₂MoO₄, 0.079 mg/L CuSO₄, and 0.049 mg/L Co(NO₃)₂, pH 7.4 and may be supplemented with 16 μg/L biotin, 20 mM MgSO₄, 8 mM KCl. Saline media, such as MBG-11, are further supplemented with 35 g/L NaCl.

In another embodiment, Synechococcus sp. strain PCC 7002 cells may be cultured on medium A supplemented with 35 g/L salt (Stevens et al., J. Phycol., 9:427-430 (1973)). Medium A is a Tris-buffered (pH 8.2) medium containing 18 g L⁻¹ NaCl, 0.6 g L⁻¹ KCl, 1.0 g L⁻¹ NaNO₃, 5.0 g L⁻¹ MgSO₄.7H₂O, 50 mg L⁻¹ KH₂PO₄, 266 mg L⁻¹ CaCl₂, 30 mg L⁻¹ Na₂ EDTA.2H₂O, 3.89 mg L⁻¹ FeCl₃.6H₂O, 1 g L⁻¹ Tris/HCl (pH 8.2), 1 mL L⁻¹ P1 trace metal solution, 4 μg L⁻¹ vitamin B₁₂. P1 trace metal solution (1000×) contains the following substances: 34.26 g L⁻¹ H₃BO₃, 4.32 g L⁻¹ MnCl₂.4H₂O, 0.315 g L⁻¹ ZnCl₂, 0.03 g L⁻¹ MoO₃ (85%), 0.003 g L⁻¹ CuSO₄.5H₂O, 0.01215 g L⁻¹ CoCl₂.6H₂O.

### Endogenous Cyanobacterial Plasmids

Wild-type cyanobacteria strains contain several different endogenous plasmids in addition to their chromosome. These endogenous plasmids are essential to the survival of the cell and are passed on to each daughter cell during cellular division. Typically, each plasmid has several copies per cell. Different plasmids vary in size. Synechococcus sp. strain PCC 7002, for example, contains six endogenous plasmids, pAQ1, pAQ3, pAQ4, pAQ5, pAQ6, and pAQ7, ranging in size from 4.8 Kb (pAQ1) to 186 Kb (pAQ7), see Table 1. The relative number of the endogenous plasmids per cell varies from a low copy number (e.g., about 15 copies or less per cell) to a high copy number (e.g., about 20 or more copies per cell). Among the six endogenous plasmids of Synechococcus sp. strain PCC 7002, pAQ1 has the highest copy number, at about 50 copies per cell and pAQ3 has about 27 copies per cell. Another endogenous plasmid, pAQ4 (32 Kb) has approximately 15 copies per cell. For comparison, the chromosomal DNA of Synechococcus sp. strain PCC 7002 has 6 copies per cell.

The terms high, low or medium as applied to copy numbers are relative to the number of copies of various endogenous plasmids in a cell. In an embodiment, a high copy number of an endogenous plasmid is greater than 20 copies per cell. In another embodiment, a high copy number of an endogenous plasmid is greater than 25 copies per cell. In another embodiment, a high copy number of an endogenous plasmid is greater than 50 copies per cell. In another embodiment, a high copy number of an endogenous plasmid is greater than 70 copies per cell. In an embodiment, a low copy number of an endogenous plasmid is less than about 15 copies per cell. In another embodiment, a low copy number of an endogenous plasmid is less than 10 copies per cell. In another embodiment, a low copy number of an endogenous plasmid is less than 5 copies per cell.

TABLE 1

| Genetic Element | Size (bp) | Genes | Copies/cell |
|---|---|---|---|
| Chromosome | 3008047 | 2872 | 6 |
| pAQ7 | 186459 | 165 | 6 |
| pAQ6 | 124030 | 109 | 6 |
| pAQ5 | 38515 | 39 | 10 |
| pAQ4 | 31972 | 30 | 15 |
| pAQ3 | 16103 | 17 | 27 |
| pAQ1 | 4809 | 3 | 50 |

Endogenous plasmids in marine cyanobacteria carry gene sequences that are essential to the cell's survival. Placing recombinant, exogenous genes onto endogenous plasmids (e.g., pAQ1, pAQ3, pAQ4, pAQ5, pAQ6, and pAQ7) can result in the recombinant genes staying in the cells longer than if they were on a foreign-derived plasmid when no antibiotic selection is used.

Without being bound by theory, the loss of foreign-derived plasmids by a transformed cyanobacteria or daughter cell can happen because the foreign-derived plasmid confers a burden to those cells that possess it. The wild-type cells that do not possess the foreign-derived plasmid will be more likely to dominate a population of cyanobacteria. Even if the foreign-derived plasmid contains a selectable marker such as antibiotic resistance, the foreign-derived plasmid is not genetically stable and can be lost over time if no antibiotic selection pressure is applied.

A high copy number of a plasmid within a cell is useful for increased transcription and translation of a protein or proteins encoded by an exogenous polynucleotide sequence on the plasmid. Each separate plasmid copy can be used as a template for the production of RNA transcripts. Thus, 50 copies of a plasmid could potentially produce 50× more RNA transcript than just one plasmid. Because an increased level of RNA transcript often correlates with an increase in the level of translation of the encoded protein, this can result in higher yields of product or products produced by the encoded protein or proteins.

The pAQ1 endogenous plasmid carries at least one gene that is essential for cell survival as well as a gene involved in high temperature acclimation (Kimura et al., Plant Cell Physiol., 43:217-223; (2002)). Thus, the pAQ1 plasmid appears to be necessary for cell survival. Among the endogenous plasmids in Synechococcus sp. strain PCC 7002, pAQ1 has the highest copy number at about 50 copies per cell. Thus, for heterologous gene expression (expression of exogenous polynucleotide sequences), the use of pAQ1 could lead to higher levels of gene expression. This can be an advantage when gene expression level is a limiting factor to the expression of the exogenous polynucleotide sequence constructs and/or cassettes.

A limiting aspect of expressing exogenous polynucleotides is the genetic stability of the exogenous polynucleotide sequence. The metabolic burden associated with expressing exogenous polynucleotide sequences imparts a counter-selective pressure on cells that contain them. Thus, a cell programmed to maintain high copy numbers of an endogenous plasmid would be at a metabolic disadvantage if it contained a high proportion of endogenous plasmids containing exogenous polynucleotide sequences (also referred to as recombinant forms of endogenous plasmids).

The method of obtaining a population of cells substantially enriched in one form of a plasmid is referred to as segregation. A fully segregated cell population will have a substantially unified proportion of one plasmid type over another. For example, a fully segregated cell population would contain only recombinant forms of endogenous plasmids and have substantially no copies of the corresponding wild-type endogenous plasmid.

Antibiotic resistance is a commonly used selection marker in genetic engineering. An antibiotic selection marker confers resistance to an antibiotic. Examples of commonly used antibiotics for selecting clones containing antibiotic resistance selection markers include, but are not limited to, streptomycin, spectinomycin, kanamycin, gentamycin, erythromycin, neomycin, rifampin, ampicillin, and zeomycin resistance. Other antibiotic resistance genes can be used. Other types of selection markers may be used, such as any marker enabling the cyanobacteria to grow on media that it would not be able to grow on but for the expression of the selection marker.

In an embodiment, an antibiotic resistance selection marker is Tn903, see Grindley, N. D., and Catherine M. Joyce. “Genetic and DNA sequence analysis of the kanamycin resistance transposon Tn903.” Proceedings of the National Academy of Sciences 77.12 (1980): 7176-7180, hereby incorporated by reference.

In an embodiment, an antibiotic resistance selection marker is aadA, see Clark, Nancye C., et al. “Detection of a streptomycin/spectinomycin adenylyltransferase gene (aadA) in Enterococcus faecalis.” Antimicrobial agents and chemotherapy 43.1 (1999): 157-160, hereby incorporated by reference.

At least because of its high copy number, it can be very difficult to achieve complete segregation of the recombinant form of an endogenous pAQ1 plasmid in Synechococcus strain sp. PCC 7002. Even with a selective marker such as antibiotic resistance on the recombinant pAQ1 plasmid or other high copy number recombinant forms of endogenous plasmids, there is a high probability that copies of endogenous pAQ1 plasmids will be transferred to daughter cells by random distribution because there will be enough recombinant plasmids to produce sufficient levels of resistance to a selective agent. An additional problem in obtaining fully segregated populations of cyanobacteria containing only endogenous recombinant pAQ1 plasmids is that the high copy number of the recombinant pAQ1 plasmids leads to higher levels of expression of a given selection marker which reduces the selection pressure on the endogenous plasmids contained within the cells. This further facilitates residual transfer of the endogenous plasmids to daughter cells.

Endogenous plasmids other than pAQ1 can also be used as recombinant forms of endogenous plasmids that encode exogenous polynucleotide sequences. For example, it is possible that, in Synechococcus sp. strain PCC 7002, any of the endogenous plasmids such as pAQ1, pAQ3, pAQ4, pAQ5, pAQ6, or pAQ7 can be used. In a one aspect, the endogenous plasmid used to encode exogenous polynucleotide sequences is pAQ1 or pAQ3.

The recombinant form of endogenous plasmids disclosed herein can also be used as an integration platform for essential or conditionally essential genes. These genes can, for example, be integrated into endogenous cyanobacterial plasmids via homologous recombination. It is also possible to create shuttle vectors by combining the backbones of endogenous plasmids and with backbones of self-replicating E. coli vectors, for example.

In one aspect, the disclosure provides vectors for genetically stable integration of an expression cassette (e.g., an ethanologenic expression cassette) into the Synechococcus sp. strain PCC 7002 endogenous plasmid pAQ1. Integration of an expression cassette into the endogenous plasmid pAQ1 may be achieved by transforming the host cyanobacterium cell with a non-replicative vectors or linear DNA fragments containing the expression cassette flanked by sequences that are homologous to sequences of the pAQ1 plasmid so that the expression cassette can be integrated into the endogenous plasmid through homologous recombination.

### Expression Cassettes

In one aspect, this disclosure provides vectors modified to include an expression cassette capable of expressing a gene of interest such as selection marker genes and ethanologenic genes. In some embodiments, the expression cassette includes an antibiotic selection marker that is a gene that encodes for a protein that confers resistance to an antibiotic. Examples of antibiotics include streptomycin, spectinomycin, kanamycin, gentamycin, erythromycin, neomycin, or zeomycin.

In other embodiments, the expression cassettes provided herein are capable of synthesizing carbon-based products of interest derived from various biosynthetic pathways by fixing CO₂. Such products are useful in the context of fuels, biofuels, industrial and specialty chemicals, additives, as intermediates used to make additional products (e.g., alcohols such as ethanol, propanol, isopropanol, isobutanol, butanol, fatty alcohols, long chain alcohols, fatty acid esters, wax esters); hydrocarbons and alkanes such as propane, octane, diesel, JP8; polymers such as terephthalate, 1,2-propanediol, 1,3-propanediol, 1,4-butanediol, polyols, PHA, PHB, acrylate, adipic acid, ε-caprolactone, isoprene, caprolactam, rubber; commodity chemicals such as lactate, DHA, 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, HPA, lactic acid, THF, gamma butyrolactone, pyrrolidones, hydroxybutyrate, glutamic acid, levulinic acid, acrylic acid, malonic acid, specialty chemicals such as carotenoids, isoprenoids, isobutyraldehyde, itaconic acid; pharmaceuticals and pharmaceutical intermediates such as 7-ADCA/cephalosporin, erythromycin, polyketides, statins, paclitaxel, docetaxel, terpenes, peptides, steroids, omega fatty acids and other such suitable products of interest).

Genes required for synthesizing carbon-based products derived from various biosynthetic pathways by fixing CO₂ are well known to those skilled in the art. For example, to create an expression cassette capable of producing ethanol (e.g., an ethanologenic cassette), the cassette is modified to express ethanologenic genes (e.g., a pyruvate decarboxylase (PDC) gene and an alcohol dehydrogenase (ADH) gene). In a further example, to create an expression cassette capable of producing butanol (e.g., a butanologenic cassette), the cassette is modified to express atoB (acetyl-CoA acetyltransferase), β-hydroxybutyryl-CoA dehydrogenase (Hbd), crotonase (Crt), butyryl CoA dehydrogenase, CoA-acylating aldehyde dehydrogenase (ALDH), and adhE encoding an aldehyde-alcohol dehydrogenase.

In various embodiments, expression cassettes containing genes encoding enzymes for production of product or products of interest are introduced into the host cell such that expression of the enzyme by the host under certain conditions results in increased production of a product of interest. In certain cases, introduction takes place through transformation of the expression cassette into the host cell. Increased production or up-regulation of a product of interest includes both augmentation of native production of the product of interest as well as production of a product of interest in an organism lacking native production. For example, in some instances production will be increased from a measurable initial value whereas in other instances the initial value is zero.

### Shuttle Vectors

A shuttle vector refers to a vector which can propagate in two different host species. DNA inserted into a shuttle vector can be tested or manipulated in two different cell types. The main advantage of these vectors is that they can be manipulated in a system having a high growth rate where the DNA is more easily manipulated (e.g., E. coli) then used in a system which is more difficult or slower to use (e.g. cyanobacteria, yeast, other bacteria). For the plasmids to be able to replicate in either type of host cell, a shuttle vector typically contains a separate nucleic acid sequence that functions as an origin of replication for each host cell type, or, alternatively, one origin of replication sequence that works in multiple species.

Examples of useful shuttle vectors that can propagate in both E. coli and cyanobacteria species include, for example, the RSF1010-based shuttle vector, as well as a pAQ1-based shuttle vector, which is derived from the endogenous pAQ1 plasmid. These vectors combine the ease of working in E. coli with the advantage of directly propagating them in cyanobacteria without further manipulation.

In one embodiment, this disclosure provides shuttle vectors derived from the endogenous cyanobacteria plasmids (e.g., pAQ1, pAQ3, pAQ4, pAQ5, pAQ6, and pAQ7). The shuttle vectors can be designed to self-replicate in both E. coli and cyanobacteria strains (e.g., Synechococcus sp. PCC 7002). To prepare the shuttle vectors, the portions of the endogenous cyanobacteria plasmids required for replication and maintenance in the cyanobacterial cell provide the backbone of the vector. In some embodiments, the entire sequence of the endogenous cyanobacteria plasmid serves as the backbone of the vector. The backbone is converted to a cyanobacteria/E. coli shuttle vector by integrating an E. coli origin of replication (e.g., pUC ori) into a portion of the backbone. To conserve the function of the endogenous plasmid, the E. coli origin of replication may be inserted into a non-essential portion of the backbone.

In another embodiment, an E. coli based plasmid containing an E. coli origin of replication and a selection marker (e.g., an antibiotic resistance gene) serves as the backbone of the shuttle vector. The backbone is converted to a cyanobacteria/E. coli shuttle vector by inserting all or a portion of an endogenous cyanobacteria plasmid into the E. coli plasmid backbone.

Shuttle vectors can also be modified to include an expression cassette capable of expressing a gene of interest such as selection marker gene and ethanologenic genes. In an embodiment, the expression cassette includes a selectable marker, for example an antibiotic selection marker. In another embodiment, the expression cassette is an ethanologenic expression cassette capable of expressing a PDC gene and an ADH gene.

### Integration Vectors

In another embodiment, the disclosure provides methods for making and using integration vectors in cyanobacteria. Integration vectors disclosed herein are useful for integrating a recombinant expression cassette (an exogenous polynucleotide sequence) into an endogenous cyanobacterial plasmid (e.g., pAQ1, pAQ3, pAQ4, pAQ5, pAQ6, and pAQ7) or chromosome through homologous recombination events.

Integration vectors of the present disclosure are prepared by integration of expression cassettes with selection markers into various selected sites of cyanobacterial endogenous plasmids. The expression cassettes may include promoters, genes expressing heterologous proteins, and various selection markers. The exogenous polynucleotide sequences, also referred to as expression elements, are flanked by nucleotide sequences that are homologous to selected regions of endogenous cyanobacterium plasmid. The flanking nucleotide sequences are designed to target integration of the expression cassette into specific sites which do not interfere with the expression of essential genes.

Integration vectors consist of an insert and a backbone. In an embodiment, the insert is an exogenous polynucleotide to be integrated into a backbone that is essentially an endogenous cyanobacterial plasmid such as pAQ1, pAQ3, pAQ4, pAQ5, pAQ6, and pAQ7, for example. The backbone of the vector may be modified to include an expression cassette flanked on both ends with nucleotide sequences that are homologous to regions of the endogenous cyanobacterial plasmid. In addition to the flanking regions, the expression cassette is designed to include a selection marker and a gene or genes of interest to be heterologously expressed (e.g., a PDC gene and/or an ADH gene). In an embodiment, the flanking regions are design to be integrated into the endogenous plasmid at sites which do not interfere with the expression of any essential gene(s).

In an embodiment, a cyanobacteria/E. coli shuttle vector, as described above, may serve as the backbone for a cyanobacterial integration vector. An expression cassette comprising a selection marker and a gene of interest to be heterologously expressed (e.g., a PDC gene and/or an ADH gene) is prepared. The expression cassette is further modified to comprise flanking regions positioned both upstream and downstream of the selection marker and a gene of interest to be heterologously expressed. The flanking regions comprise nucleotide sequences that are homologous to regions of an endogenous cyanobacterial plasmid to achieve targeted integration of the expression cassette. The expression cassette, with the corresponding flanking regions, is then integrated into the backbone of the cyanobacteria/E. coli shuttle vector, such that the integrated cassette does not interfere with any essential elements of the shuttle vector.

Successful integration of an expression cassette allows for its selection for single recombination integration events when transformed as a circular plasmid. Alternatively, the integration vector may be linearized by restriction enzyme digestions, either single or double, to isolate the sequence to be integrated and transformed via natural transformation. During this linearization process, unnecessary DNA sequences (such as, for example, the E. coli origin of replication of the backbone of the integration vector) can be removed, making the final vector smaller, providing improved transformation efficiency. The DNA fragment can also be generated by PCR amplification.

Both long exogenous polynucleotide segments and negative selection pressure can reduce the efficiency of integration. In one aspect, the present disclosure provides for integration sites on endogenous pAQ1 plasmid that allow for more efficient, and thus more probable, integration of DNA. In an embodiment, integration vectors of the present disclosure are designed such that integration of exogenous polynucleotide sequences into pAQ1 does not disrupt normal pAQ1 function in the cell.

### Natural Transformation of Cyanobacteria

Natural transformation of Synechococcus sp. strain PCC 7002 cells with both replicative plasmid vectors (e.g., shuttle vectors, or non-linearized integration vectors) or non-replicative vectors (e.g., linear DNA fragments) is achieved by mixing vector DNA with cells during the exponential phase of growth. In an embodiment, natural transformation can take place through conjugation.

### Full Segregation of Recombinant Forms of Endogenous Plasmids in Marine Cyanobacteria

The use of endogenous plasmids that contain at least one gene essential for the survival of the host cell as expression vectors containing exogenous polynucleotide sequences is an effective strategy for creating a stable expression system in cyanobacteria. Cyanobacteria transformed with a recombinant form of an endogenous plasmid initially contain both the recombinant form of an endogenous plasmid and the wild-type endogenous plasmid. A recombinant form of an endogenous plasmid will be maintained in the cell and daughter cells under selective pressure, for example growth on a medium containing an antibiotic when the selection marker is an antibiotic resistance gene. The recombinant form of an endogenous plasmid will compete with the wild-type endogenous plasmid for survival in the cell. This could result in the recombinant form of an endogenous plasmid being out-competed by the original endogenous plasmid when no antibiotic selection is applied to the culture. Thus, without full segregation of cells containing only the recombinant form of an endogenous plasmid and no corresponding wild-type endogenous plasmids, the recombinant form of an endogenous plasmid will be lost or substantially diluted over time, resulting in reduced expression of the exogenous polynucleotide sequence carried by the recombinant form of an endogenous plasmid. Thus, the products of proteins encoded for therein will decrease over time if the recombinant cell population is not fully segregated.

Growing cyanobacteria in a selective growth media can be prohibitively expensive for large scale growth in bioreactors used for production of biofuels or other compounds of interest and also raises several environmental, safety, and regulatory issues. Thus, for efficient and stable expression of exogenous polynucleotide sequences carried by the recombinant forms of endogenous plasmids within cyanobacterial cells, it is useful to fully segregate clones which contain copies of the recombinant form of endogenous plasmids and which do not maintain any copies of the corresponding wild-type endogenous plasmid from clones which contain mixed populations of endogenous recombinant and wild-type endogenous plasmids.

The usual medium for growth of marine cyanobacterial strains, including Synechococcus sp. strain PCC 7002, is BG-11 supplemented with about 35 g NaCl/L (1×), also known as MBG-11. This high salt/saline culture medium creates some difficulties when using antibiotic resistance genes to select for recombinant cells. In these high salt cultures, the minimum inhibitory concentration (“MIC”) of a given antibiotic is much higher than in freshwater cultures. The higher MIC causes antibiotics to precipitate out of solution before they reach useful concentrations for selection. Problems caused by the increased MIC are further exacerbated when multiple copies of recombinant forms of endogenous plasmids exist in each cell because even higher concentrations of antibiotics are necessary to fully segregate cells containing only copies of recombinant forms of endogenous plasmids from cells containing mixed populations of both recombinant forms of endogenous plasmids and the corresponding wild-type endogenous plasmid because more of the antibiotic resistance protein is being expressed.

About 20-30× greater than the MIC of antibiotic is typically used when screening for recombinant plasmids containing the antibiotic resistance gene cultured in a saline medium. These concentrations of antibiotic can be so high as to not be practical because at such high concentrations the antibiotic may cause other, undesirable, side effects on the cells and/or the antibiotic may precipitate out of the medium.

Thus, in an embodiment, methods for obtaining fully segregated marine cyanobacterial cell populations are disclosed in which the antibiotic selection steps use low salt/freshwater growth media such as, BG-11. As a result of the decreased salt concentration, a much lower MIC of antibiotic is needed for full segregation. The low salt medium allows for selection using an antibiotic concentration that is about 20-30× the MIC, thus providing conditions to achieve full segregation of populations of cyanobacteria containing only copies of recombinant forms of endogenous plasmids from populations of cyanobacteria containing mixed populations of both recombinant forms of endogenous plasmids and the corresponding wild-type endogenous plasmid. In another embodiment, 1×, 2×, 5×, 10×, 20×, 25×, 30×, 40×, 50×, 100× or more of the MIC of the antibiotic or minimum useful concentration of another selection agent can be used to achieve full segregation.

In an embodiment, the antibiotic selection step includes culturing a transformed marine cyanobacteria cell on a low salt medium containing a concentration of antibiotic or other selection agent sufficient for full segregation. The cyanobacterial cells may be cultured in liquid media and on agarose plates. In an embodiment, cultures are maintained at 28° C. to 37° C. for a period of 7 to 14 days with continuous light. In an embodiment, low salt media is a growth media such as BG-11, or other media having low salt (e.g., less than about 15 g/L salt, less than 10 g/L salt, less than 5 g/L salt, or essentially 0 g/L salt (e.g., salt-free media). In an embodiment, a high salt media is a growth media supplemented with at least 35 g/L salt (e.g., at least 35 g/L salt, at least 40 g/L salt, at least 50 g/L salt, at least 75 g/L salt, or at least 85 g/L salt).

One of skill in the art will recognize that the type and amount of antibiotic agent is limited only in that the antibiotic agent correspond with the selection marker to which the recombinant endogenous plasmid confers resistance. A sufficient amount of the antibiotic agent means an amount to be effective for killing or inhibiting the growth of any host cell that does not maintain the selection marker and may depend on the particular antibiotic, the selection marker, and/or the growth conditions. The amount of antibiotic constituting a “sufficient amount” can be determined by one of ordinary skill through routine experimentation.

In an embodiment, the transformed marine cyanobacterial cells containing recombinant forms of endogenous plasmids can be first grown in a high salt medium containing a selective agent and then are fully segregated in a low salt medium containing a sufficient amount of antibiotic or other selection agent to fully segregate the transformed marine cyanobacteria that contain only recombinant forms of the endogenous plasmid and that do not contain wild-type forms of the corresponding endogenous plasmid.

In an embodiment, the transformed marine cyanobacterial cells containing recombinant forms of endogenous plasmids are first cultured in a high salt medium containing a selective agent and then are plated on low salt agarose plates containing amounts of a selective agent ranging from about 1×MIC to about 100×MIC or more. In an embodiment, transformed marine cyanobacterial cells are streaked for single colonies on low salt agarose-containing media, e.g. plates, with 1×, 2×, 5×, 10×, 20×, 25×, 30×, 40×, 50×, 100× or more of the MIC of a given antibiotic selection agent or other selection agent. For example, a high salt media containing marine cyanobacterial cells transformed with a recombinant form of an endogenous plasmid containing a spectinomycin resistance marker gene may be plated onto low salt agarose plates variously containing 15 μg/mL, 30 μg/mL, 60 μg/mL, 120 μg/mL 240 μg/mL, 320 μg/mL, 400 μg/mL and 500 μg/mL spectinomycin.

In an embodiment, the plated, low salt agarose plates variously containing different amounts of selective agent are then cultured for a sufficient time to allow for the growth of colonies. Single colonies are then selected from the plate or plates that contain at least one single colony. Single colonies can be selected from plates that contain multiple isolated single colonies. These single colonies are then restreaked on low salt agarose plates containing about the MIC fold concentration of the selective marker from which they were selected and cultured. The plasmid DNA of the cells that grew on the plates may then be analyzed by PCR, or other methods, for the presence of the recombinant form of the endogenous plasmid and the wild-type endogenous plasmid.

In another embodiment, transformed marine cyanobacterial cells are transferred from a high salt growth medium to low salt agarose plates, or media, containing 1× of the MIC of the antibiotic selection agent and then transferred to plates with successively higher concentrations of antibiotic selection agent. For example, transformed marine cyanobacterial cells are then transferred to plates containing 2× of the MIC of the antibiotic selection agent to 5× to 10× to 20× to 30× to 40× to 50× to 100× of the MIC antibiotic selection agent. For example, a marine cyanobacterial cell transformed with a recombinant form of an endogenous plasmid containing a spectinomycin resistance marker gene may be successively transferred from low salt agarose media plates containing 15 μg/mL spectinomycin to 30 μgi/mL spectinomycin to 60 μg/mL to 120 of μg/mL to 240 μg/mL to 320 μg/mL to 400 μg/mL and then to plates containing 500 μg/mL spectinomycin.

In an embodiment, the method of obtaining fully segregated marine cyanobacterial clones further includes confirming that the selected clones maintain the selection marker as part of the recombinant form of an endogenous plasmid, by using PCR analysis to amplify a polynucleotide sequence specific to the recombinant form of an endogenous plasmid. For example, if the exogenous polynucleotide sequence on the recombinant form of an endogenous plasmid was an expression cassette for PDC and ADH, primers would be designed to amplify the cassette through PCR. The PCR products would be analyzed by gel chromatography to verify the presence of a product corresponding to the size of the polynucleotide sequence encoding the PDC/ADH expression cassette. In another embodiment, other methods are used to test for the presence or absence of the wild-type form of the endogenous plasmid and/or the recombinant form of the endogenous plasmid.

In an embodiment, the method of obtaining fully segregated marine cyanobacterial clones further includes confirming that the fully segregated cyanobacteria cells stably maintain the recombinant form of an endogenous plasmid. This is achieved by first culturing the transformed cells a high salt media containing at least about 35 g/L salt in the absence of selective pressure. Under these conditions, if the recombinant form of an endogenous plasmid is unstable and not essential for growth of the transformed cell, it will be lost. To identify marine cyanobacteria that stably maintain a given recombinant form of an endogenous plasmid, transformed cells are transferred from a medium without selective pressure to a medium with a sufficient amount of a selective agent, such as an antibiotic agent. Transformed cells which are then capable of proliferating on media containing the selective agent are identified as stably maintaining the recombinant form of an endogenous plasmid.

### Methods of Making Ethanol

The major pathway for ethanol biosynthesis is catalyzed by two enzymes, PDC and ADH. The introduction of a PDC and ADH containing ethanologenic cassette carried on a recombinant form of an endogenous plasmid into cyanobacteria enables light driven production of ethanol. PDC catalyzes the non-oxidative decarboxylation of pyruvate which produces acetaldehyde and carbon dioxide. Acetaldehyde is then converted to ethanol by ADH. Ethanol production is improved using fully segregated marine cyanobacteria that contain a recombinant form of an endogenous plasmid encoding a PDC/ADH expression cassette. The process for isolating a fully segregated recombinant cyanobacteria clone may be conducted according to the methods described herein. Fully segregated clones can be maintained in culture (with or without an antibiotic agent) for an extended period of time, up to 5 months or more, for example, and therefore exhibit improved ethanol production characteristics in continuous culture when compared to clones having mixed, or not fully segregated populations.

Production of ethanol using recombinant marine cyanobacteria depends on the culture conditions used, such as the composition of the medium, temperature, CO₂ concentration, and duration of light exposure. In an embodiment, fully segregated marine cyanobacteria containing recombinant forms of endogenous plasmids encoding a PDC/ADH ethanologenic cassette are grown in a medium (e.g., MBG-11, medium A or medium A+ supplemented with 35 g/L salt). The cultures are then maintained at 28° C. to 37° C. and bubbled continuously with 5% CO₂.

### EXAMPLES

### Example 1

### Construction of a Shuttle Vector Based on Endogenous Plasmid pAQ1

Shuttle vectors derived from an endogenous cyanobacterial plasmid pAQ1 as the “backbone” were prepared, see Table 2. The shuttle vectors were designed to self-replicate in both E. coli and in cyanobacteria. The vectors are easily manipulated in E. coli, such that recombinant genes and any other desired DNA sequences can be placed onto the vector. The shuttle vectors can also be transformed into the cyanobacterial cells.

TABLE 2

| Shuttle | | |
|---|---|---|
| Vector | Selection | |
| Designation | Marker | Recombinant Genes/Pathways |
| pAB408 | Amp^{R} | N/A (empty vector) |
| pAB409 | Gm^{R} | N/A (empty vector) |
| pAB413 | Sp^{R} | N/A (empty vector) |
| pAB218 | Sp^{R} | PrbcL(6803)-PDC-ADH |
| pAB232 | Sp^{R} | PnblA(7120)-PDC-PrbcL(6803)-ADH |
| pAB410 | Gm^{R} | PcpcB(7002)-PDC-ADH |
| pAB414 | Sp^{R} | PcpcB(7002)-PDC-ADH |
| pAB416 | Gm^{R} | PrbcL(6803)-PDC-ADH |
| pAB417 | Gm^{R} | PnblA(7120)-PDC-ADH |
| pAB418 | Gm^{R} | PggpS(7002)-PDC-ADH |
| pAB422 | Sp^{R} | PrbcL(6803)-PDC-ADH |
| pAB423 | Sp^{R} | ZiaR-PziaA(6803)-PDC-ADH |
| pAB424 | Sp^{R} | PsmtA(7002)-PDC-ADH |
| pAB425 | Sp^{R} | PcpcB(6803)-PDC-ADH |
| pAB220 | Sp^{R} | PdpsA1(7002)-PDC-ADH |
| pAB224 | Sp^{R} | PA1478(7002)-PDC-ADH |
| pAB225 | Sp^{R} | PmscA(7002)-PDC-ADH |

Three empty shuttle vectors were constructed from the pAQ1 endogenous plasmid of Synechococcus sp. strain PCC 7002. One, designated pAB409, contains a gentamycin resistance (Gm^{R}) selection marker. The second, designated pAB413, contains a streptomycin/spectinomycin resistance (Sp^{R}) selection marker. The third, designated pAB408, contained an ampicillin resistance (Amp^{R}) selection marker. These pAQ1 shuttle vectors retain the entire sequence of the endogenous plasmid pAQ1. In an embodiment, an ethanologenic gene cassette is cloned into the empty vector so that ethanol can be produced.

The first empty shuttle vector, designated pAB409, was constructed by inserting a PCR amplified pAQ1 into backbone vector pAB303, which is derived from pCR-Blunt II-TOPO (Invitrogen), and contains a pUC ori and a gentamycin resistance gene (i.e., a gentamycin selection marker). The pAQ1 was amplified using primers “pAQ1 Pst fwd” (CGTCGACTGCAGCCTTAACTCACTGTGATCGT) and “pAQ1Xba rev” (CGTCGATCTAGACCTCCTTCGCCTGACGATC) and used PCC 7002 genomic DNA as a template. The amplified pAQ1 was then digested with PstI and XbaI and ligated into vector pAB303, which was digested with the same enzymes, creating pAB409.

The second empty shuttle vector, designated pAB413, was constructed by inserting a PCR amplified pAQ1 into backbone vector pAB304, which is derived from pCR-Blunt II-TOPO (Invitrogen), and contains a pUC ori and a spectinomycin resistance gene (e.g. a spectinomycin selection marker). The pAQ1 was amplified using primers “pAQ1 Pst fwd” (CGTCGACTGCAGCCTTAACTCACTGTGATCGT) and “pAQ1 Xho rev” (CGTCGACTCGAGCCTTAACTCACTGTGATCGT) and used as template genomic DNA isolated from PCC 7002. The amplified pAQ1 was then digested with PstI and XhoI and ligated into vector pAB304, which was digested with the same enzymes, creating pAB413.

The third empty shuttle vector, plasmid pAB408 is the entirety of pAQ1 and the genes in the backbone vector pUC57 are in the same direction as the genes in the pAQ1 plasmid. Plasmid pAB408 was also constructed so that the pUC57 backbone would not interfere with the reported putative genes in plasmid pAQ1.

Next, shuttle vectors containing an expression cassette of PDC and/or ADH genes were prepared. Plasmids pAB410 and pAB414 were constructed by inserting a PCR amplified PcpcB-PDC-ADH cassette into backbone vectors pAB409 and pAB413, respectively. The PcpcB-PDC-ADH cassette was amplified using a plasmid preparation of pAB405 (a RSF11010-based shuttle vector: p309-PcpcB-PDC-ADH-Spc/Gm) as a template. PCR amplification was done using primers “CpcB SpeI SalI fwd” (CGTGGACTAGTTCACGGTCGACATCCTCCCAGGAAA) and “405 ADH Pst rev” (ATGCTCTTCTGCTCCTGCAG). This cassette was then digested with SpeI and PstI and ligated into the respective vector which was then digested with the same enzymes to create pAB410 and pAB414.

Other shuttle vectors were constructed by digesting the appropriate vector (either pAB410 or pAB414) with SalI and EcoRI. The appropriate promoter was PCR amplified and digested with Sail and EcoRI. The PCR amplified promoter was the ligated into the appropriate vector.

For the vectors described in Table 2, PrbcL(6803)-PDC-ADH refers to an ethanologenic cassette where the Zymomonas mobilis PDC and Synechocystis ADH genes are driven by the rbcL promoter from Synechocystis strain PCC 6803. Pnb1A(7120)-PDC-PrbcL(6803)-ADH refers to an ethanologenic cassette where the PDC gene is driven by the nb1A promoter from Nostoc sp. PCC 7120 and the ADH gene is driven by the rbcL promoter from Synechocystis strain PCC 6803. PcpcB(7002)-PDC-ADH refers to an ethanologenic cassette where the PDC and ADH genes are driven by the cpcB promoter from Synechococcus PCC 7002. PcpcB(6803)-PDC-ADH refers to an ethanologenic cassette where the PDC and ADH genes are driven by the cpcB promoter from Synechocystis strain PCC 6803. Pnb1A(7120)-PDC-ADH refers to an ethanologenic cassette where the PDC and ADH genes are driven by the nb1A promoter from Nostoc sp. PCC 7120. PggpS(7002)-PDC-ADH refers to an ethanologenic cassette where the PDC and ADH genes are driven by the ggpS promoter from Synechococcus PCC 7002. ZiaR-PziaA(6803)-PDC-ADH refers to an ethanologenic cassette where the PDC and ADH genes are driven by the ZiaR-PziaA promoter system from Synechocystis strain PCC 6803. PsmtA(7002)-PDC-ADH refers to an ethanologenic cassette where the PDC and ADH genes are driven by the smtA promoter from Synechococcus PCC 7002. PdpsA1(7002)-PDC-ADH refers to an ethanologenic cassette where the PDC and ADH genes are driven by the dpsA1 promoter from Synechococcus PCC 7002. PA1478(7002)-PDC-ADH refers to an ethanologenic cassette where the PDC and ADH genes are driven by the PA1478 promoter from Synechococcus PCC 7002. PmcsA(7002)-PDC-ADH refers to an ethanologenic cassette where the PDC and ADH genes are driven by the mcsA promoter from Synechococcus PCC 7002.

### Example 2

### Construction of Integration Vectors Based on Endogenous Plasmid pAQ1

The design of the vector for integration allows for its selection for single recombination integration events when transformed as a circular plasmid. However, preferably, the vector is linearized by double digestions to isolate the sequence to be integrated and transformed via natural transformation, selecting for double crossover recombination events. During this linearization process, unnecessary DNA sequences (such as, for example, the pUC origin) can be removed, making the final plasmid shorter. The linear DNA fragments to be integrated may also be prepared by PCR amplification.

A umber of pAQ1-based integration vectors were made containing different combinations of flanking regions. They were designed to increase the efficiency of transformation as well as to increase the probability of integration of the vectors. The pAQ1-based integration vectors were then used in the methods disclosed herein to achieve full segregation of transformed cell populations. The design of these vectors allows for easy replacement of any component of the integration vector, so that different integration sites can be targeted, the efficiency of other promoters can be assayed, and alternative selection markers can be added to select for double recombination events.

Methods for constructing integrative plasmids used in the methods disclosed herein can generally be found in Xu et al. (Methods Mol. Biol. 684:273-293; 2011). Xu et al. describe the use of the endogenous plasmid pAQ1 for transformation of Synechococcus sp. strain PCC 7002 and for integration of exogenous polynucleotide sequences. Xu et al. do not use their integration vectors in the methods for achieving full segregation described herein. Xu et al. describe an integration vector including an expression cassette integrated between two regions of homology, designated “Flank A” and “Flank B”. Similar work to Xu et al. is described in the thesis of Byrne, P. O. (2010 Undergraduate Thesis. “Differential and inducible expression of yellow fluorescent protein in the marine cyanobacterium Synechococcus sp. PCC 7002” Pennsylvania State University Press).

pAB104: Plasmid pAB104 was constructed, see FIG. 1, and contains two regions of homology, Flank A and Flank B, as described by Xu et al. pAB104 was constructed using pAB503 as the backbone vector. pAB503 is derived from plasmid pCR-Blunt II-TOPO and contains a spectinomycin (Sp^{R}) selection marker gene flanked by the upstream and downstream sequences of a cyanobacterial gene of interest to achieve targeted gene interruption. The vector contains restriction sites for the easy substitution of the flanking sequences so that gene integration or deletion can be performed at different sites on plasmids or on chromosomes. Flank A and Flank B of pAQ1 were generated by PCR using primers pAQ1 Flank AF (5′-AAGGCACTGCAGCTTTCTCTTATGCACAGATGGG-3′) and pAQ1 Flank AR (5′-TTTGGCCTCGAGGGGGTTTTCTCGTGTTTAGGC-3′) for Flank A, and pAQ1 Flank BF (5′-AACAGGGGATCCCTCTCACCAAAGATTCACCTG-3′) and pAQ1 Flank BR (5′-AAAAGTCGACATATATACTAGTCTAAGCCTCCTGAATAAATCTATTTATAC-3′) for Flank B.

Flank A was digested with PstI and XhoI and ligated into pAB503. The resulting plasmid was then digested with BamHI and SpeI and ligated with Flank B which was digested with the same enzymes to create pAB104. This vector enables gene integration into pAQ1 via double crossover homologous recombination, which allows for efficient integration via natural transformation.

pAB105: pAB105 was constructed using the PcpcB(7002)-PDC-ADH cassette isolated from plasmid pAB401 (a RSF1010-based shuttle vector: pSA423-PcpcB-PDC-ADH-Spc) by digestion with SalI and PstI. The fragment of the ethanologenic cassette was ligated to the integrative vector pAB104 digested with the same enzymes to create plasmid pAB105.

pAB106: pAB106 was constructed by ligating PCR generated promoter sequence PggpS(7002) that was digested with SalI and EcoRI to pAB105 that was also digested with the same enzymes. As a result, the ethanologenic cassette in pAB106 is PggpS(7002)-PDC-ADH.

pAB107: pAB107 was constructed by ligating PCR generated PsigE(7002) that was digested with SalI and EcoRI to pAB105 that was digested with the same enzymes. As a result, the ethanologenic cassette in pAB107 is PsigE(7002)-PDC-ADH.

pAB108: pAB108 was constructed by ligating PCR generated PrbcL(7002) that was digested with SalI and EcoRI to pAB105 that was digested with the same enzymes. As a result, the ethanologenic cassette in pAB108 is PrbcL(7002)-PDC-ADH.

pAB109: pAB109 was constructed by ligating PCR generated PrnpA(7002) digested with SalI and EcoRI to pAB105 that was digested with the same enzymes. As a result, the ethanologenic cassette in pAB109 is PmpA(7002)-PDC-ADH.

pAB110: pAB110 was constructed by inserting a PCR generated PrbcL(6803)-PDC-ADH into pAB104. The ethanologenic cassette PrbcL(6803)-PDC-ADH was obtained by PCR-amplification from pTK020 (a RSF1010-based shuttle vector containing the ethanologenic cassette). PCR was carried out using primers TK20SynADH-Sal-F (5′-GATATCGGTCGACTTCGACATCAGGAA-3′) and TK20SynADH-Pst-R (5′-TGCTCCTGCAGATCGTGTCAAGGCTTTCCAGA-3′). The PCR product was digested with SalI and PstI and was ligated into pAB104 digested with the same enzymes.

pAB111: pAB111 was constructed by digesting p832 with PstI and EcoRI to generate the PDC-oop-PrbcL6803-synADH(deg)oop cassette. Plasmid p832 is an RSF1010-based shuttle vector that contain an ethanologenic cassette PDCoop-PrbcL6803-ADH(deg)oop cassette, where oop refers to bacteriophage lambda oop gene transcription terminator, and ADH(deg) refers to a Synechocystis ADH coding sequence, for example ADH from Synechocystis sp. strain PCC 6803, synthesized with degenerated codons. The ethanologenic cassette sequence was ligated with pAB105 digested with the same enzymes to create pAB111. As a result, the complete description of the ethanologenic cassette in pAB111 is PcpcB(7002)PDC-oop-PrbcL(6803)ADH(deg)oop.

pAB112: pAB112 was constructed by digesting p832 with PstI and EcoRI to generate the PDCoop-PrbcL6803-synADH(deg)oop cassette. The PDCoop-PrbcL6803-synADH(deg)oop cassette was ligated with pAB108 digested with the same enzymes to create pAB112. As a result, the ethanologenic cassette in pAB112 is PrbcL(7002)-PDC-oop-PrbcL(6803)ADH(deg)oop.

pAB114 (pAQ1-3): To increase the efficiency of transformation and integration of the vectors and to achieve complete segregation, a new integration vector was designed. For this new vector, designated pAB114 (FIG. 2, the flanking regions Flank A and Flank B of pAB104 were exchanged with new flanking regions, Flank A3 (SEQ ID NO: 1) and Flank B3 (SEQ ID NO: 2), see FIG. 3 and FIG. 4, respectively. The putative promoter for ORF64 was kept intact. By design, the multiple cloning sites and ethanologenic cassette was inserted between positions 3095 and 3133 of the pAQ1 plasmid. The flanking sequences are Flank A3 (positions 3133-3817 bp) and Flank B3 (positions 2469-3095 bp). The new flanking regions were PCR-amplified from DNA isolated from Synechococcus sp. strain PCC 7002 using the primers listed in Table 3. Primers used to generate Flank A3 were primers AQ1-FA3-F and AQ1-FA3-R. Primers used to generate Flank B3 were primers AQ1-FB3-F/AQ1-FB3-R.

TABLE 3

| Primer | Sequence |
|---|---|
| AQ1-FA3-F | CCGCTTTGCTCTGCAGGTAGCCCCTAGACTGTG |
| AQ1-FA3-R | GGGCGATCGCCCTCGAGATTTCCCCGCACTCG |
| AQ1-FB3-F | GTTAACTGGGATCCCTGAGCTTGTCGAGAAAGATG |
| AQ1-FB3-R | GGCATAACTAGTCCCGATACCGCAGAGCAAATC |

The product for Flank B3 was digested with BamHI and SpeI, and the product for Flank A3 was digested with PstI and XhoI. pAB104 was digested with BamHI and SpeI, and this was ligated with Flank B3 that was digested with the same enzymes. The construct containing Flank B3 was digested with PstI and XhoI, and then ligated with Flank A3 digested with the same enzymes. These ligations created the empty backbone integration construct, pAB114 as depicted in FIG. 2.

Using pAB114, several derivative plasmids were created, one of which was pAB125. pAB125 contains an ethanologenic cassette Pnb1A7120-PDC-oop-Prbc6803-ADHdeg-oop. Both pAB114 and its derivatives demonstrated high transformation efficiency. The resulting ethanologenic hybrid strains, particularly strain PCC7002:AB125, were able to make significant amounts of ethanol.

pAB124: The ethanol cassette to be used with this integration vector was isolated from plasmid pAB110. This ethanologenic cassette contained PDC and synADH(deg) under the control of two promoters and two terminators, promoter PrbcL(7002) for the PDC gene and promoter PrbcL(6803) for the ADH gene. In order to make pAB124, plasmid pAB110 was digested with SalI and PstI, and the ˜3 kb fragment was ligated into pAB114 digested with the same enzymes.

pAB125: To construct pAB125, the promoter of nb1A from Nostoc sp. PCC 7120, designated Pnb1A(7120), was PCR amplified with primers that introduced SalI and EcoRI restriction sites into the 5′ and 3′ ends, respectively. The PCR product was digested with SalI and EcoRI, and was then ligated into pAB124 that had also been digested with SalI and EcoRI. The resulting plasmid pAQ125 (FIG. 5) contained the ethanologenic cassette with PDC under the control of Pnb1A7120 and synADH(deg) under the control of PrbcL6803.

Examples of integration vectors suitable for integration into the endogenous cyanobacterial plasmid pAQ1 are provided in Table 4.

TABLE 4

| Integration | | | |
|---|---|---|---|
| Vector | Sequence of flanking | Selectable | |
| Designation | regions* | Marker | Added Recombinant Genes/Pathways |
| pAB104 | Flank A and B | Sp^{R} | Empty integration vector with Flank A |
| | | | and Flank B as in Xu et al. |
| pAB105 | Flank A and B | Sp^{R} | PcpcB(7002)-PDC-ADH |
| pAB106 | Flank A and B | Sp^{R} | PggpS(7002)-PDC-ADH |
| pAB107 | Flank A and B | Sp^{R} | PsigE(7002)-PDC-ADH |
| pAB108 | Flank A and B | Sp^{R} | PrbcL(7002)-PDC-ADH |
| pAB109 | Flank A and B | Sp^{R} | PrnpA(7002)-PDC-ADH |
| pAB110 | Flank A and B | Sp^{R} | PrbcL(6803)-PDC-ADH |
| pAB111 | Flank A and B | Sp^{R} | PcpcB(7002)-PDCoop-PrbcL(6803)- |
| | | | synADH(deg)oop |
| pAB112 | Flank A and B | Sp^{R} | PrbeL(7002)-PDCoop-PrbcL(6803)- |
| | | | synADH(deg)oop |
| pAB114 | Flank A3 and B3 | Sp^{R} | Empty vector |
| pAB124 | Flank A3 and B3 | Sp^{R} | PrbcL(7002)-PDCoop- |
| | | | PrbcL(6803)synADH(deg)oop |
| pAB125 | Flank A3 and B3 | Sp^{R} | PnblA(7120)-PDCoop- |
| | | | PrbeL(6803)synADH(deg)oop |
| pAB126 | Flank A3 and B3 | Sp^{R} | PdspA1-PDCoop-synADH(deg)oop |

### Example 3

### Natural Transformation of pAQ1-Based Shuttle Vector or Linear DNA Fragments into Synechococcus sp. Strain PCC 7002

Liquid medium A (50 mL) was inoculated with wild-type Synechococcus sp. strain PCC 7002 from 7-10 day old cultures grown in MBG-11 supplemented with vB12. The culture was grown for 3-4 days at 37° C. (12 hours light/12 hours dark) until OD₇₅₀ reached between ˜0.3-0.4. The culture was centrifuged for 10 minutes (2,500×g) at room temperature and the pellet was re-suspended in 5 mL of 5 mM EDTA. The resuspended pellet was again centrifuged for 10 minutes (2,500×g) at room temperature and the pellet was resuspended in 10 mL of fresh Medium A to an OD₇₅₀˜1.5-2.0. From this mixture, 2 mL aliquots were transferred to vented 50 mL bioreactor tubes. A total of 5 μg of the DNA vector was added to each 2 mL cell suspension and incubated for 5-6 hours under high light conditions with shaking at 37° C. After incubation, 2 mL of fresh medium A was added to each cell suspension; and incubated under low light, with shaking at 28° C. for 2 days as a recovery period.

Following the recovery period, 0.3 mL of cell suspension was plated onto a BG-11 supplemented with vB12 plate with an antibiotic (spectinomycin at 15 mg/L or gentamycin at 2 mg/L). Transformant colonies typically took 7-10 days to appear. Colonies were tested for successful transformants by PCR analysis using forward and reverse primers specific to the transformed recombinant vector.

### Example 4

### Transformation of Integration Vectors

Synechococcus sp. strain PCC 7002 cells were transformed with linear DNA fragments obtained from pAQ1-based integration vectors, see Table 5. Two different types of integrative vectors were designed and constructed. Details of vector designs are provided below.

TABLE 5

| Source | Vector Designation | # of transformants |
|---|---|---|
| pAQ1-A/B | pAB104 | ~260/plate |
| (based on | (empty vector) | |
| Xu et al, 2011) | | |
| | pAB105 (PcpcB(7002)-PDC-ADH) | 0 |
| | pAB106 (PggpS(7002)-PDC-ADH) | 0 |
| | pAB107 (PsigE(7002)-PDC-ADH) | 0 |
| pAQ1-A3/B3 | pAB114 | ~1700/plate |
| | pAB124 (PrbcL(7002)-PDCoop- | 97/3 plates |
| | PrbcL(6803)synADH(deg)oop) | |
| | pAB125 (PublA(7120)-PDCoop- | 8/3 plates |
| | PrbcL(6803)synADH(deg)oop) | |
| | pAB126 (PdpsA(7002)-PDC- | 49/3 plates |
| | PrbcL(6803)-ADHdeg) | |

Table 5 depicts the transformation efficiency obtained for Synechococcus sp. strain PCC 7002 using different integrative vectors designed with integration sites pAQ1-A/B or integration sites pAQ1-A3/B3. The numbers of colonies from one or from three plates are shown. Table 5 demonstrates the effect of integrative vector design on transformation efficiency.

For the integrative vector described by Xu et al., about 1100 bp of the native plasmid was deleted, including orf64 and orf71 (pAB104, pAB105, pAB106, and pAB107). Using the Xu et al. design, transformants were generated with the empty vector (pAB104). However, no transformants were obtained using this vector containing an ethanologenic cassette (pAB105, pAB106, and pAB107). Table 5 further demonstrates that transformation efficiency was vastly improved for the new integrative vector pAQ1-B3/A3 design compared with the Xu et al. vector design.

The new integrative vector pAQ1-B3/A3 design is very different from the Xu et al. vector design. First, the precise site of integration is different. Second, the flanking sequences cloned for homologous DNA recombination are from different regions of the plasmid. Third, compared to the deletion of 1.1 kB in Xu et al. design, only 37 nucleotides in an intergenic region are deleted from the plasmid for primer designs as an effort to minimize the impact of modification on the integrity and functionality of the plasmid.

FIG. 6 depicts a map of the pAQ1 endogenous plasmid from Synechococcus sp. strain PCC 7002 showing the locations of the flanking regions used to generate integration vectors. Flank A (positions 4196-4707 bp) and Flank B (positions 2635-3015 bp) were based on Xu et al. Flank A3 (positions 3133-3817 bp) and Flank B3 (positions 2469-3095 bp) were designed for a new integrative vector that showed higher transformation efficiency.

The new integrative vector pAQ1-B3/A3 design allowed generation of stable integrants and this also helps in achieving full segregation of the transformants using the low salt segregation method. For strain PCC7002:AB125, eight total colonies were isolated from three plates. All eight colonies were confirmed to be true transformants using PCR.

Four PCC7002:AB125 transformants, which had not gone through plasmid segregation, were cultured in MBG-1 supplemented with vB12 and 100 ug/mL spectinomycin for approximately 1 week and tested for ethanologenesis using a GC-vial assay. Cultures were placed in GC vials at OD of about 1.0 and incubated at 37° C. for 24 h under continuous light of 320 μEm⁻² s⁻¹. All four clones produced ethanol at similar levels, on average 0.073% (v/v).

### Example 5

### Segregation Attempt in High Salt

Synechococcus sp. strain PCC 7002 cells were transformed with pAQ1-based shuttle vectors pAB220, pAB224, and pAB225 prepared according to Example 3. The Synechococcus sp. strain PCC 7002 transformants, each with a pAQ1-based shuttle vector, were grown in liquid MBG-11 medium supplemented with vB12 for 4 days to about 1 week and transferred successively to medium with increasing concentrations of antibiotic (increasing from spectinomycin level of 100 mg/L to 800 mg/L), and cultured on each medium for 4 to 7 days. Cultures were finally plated on MBG-11 plates containing a high level of antibiotic (800 mg/L spectinomycin).

Cells from different clones were extracted to prepare DNA to be used as template in PCR reactions. Primers pAQ1flankAFnew, specific to Flank A sequence, and pAQ13939R, specific to Flank B sequence, were used, see Table 6. Wild-type pAQ1 would yield an expected PCR product of 1.2 kb. pAQ1-based recombinant shuttle vectors containing different promoter-PDC-ADH cassettes would be expected to produce a product of about 7 kb.

FIG. 7 is an image of an ethidium bromide-stained gel demonstrating PCR analysis following segregation attempts of Synechococcus sp. strain PCC 7002 transformed with pAQ1-based shuttle vectors pAB220, pAB224 and pAB225. Lane assignments are as follows: (A) H₂O control; (B) PCC7002 WT; (C) PCC7002:pAB220 conjugation clone 2; (D) PCC7002:pAB220 conjugation clone 4; (E) PCC7002:pAB224 conjugation clone 1; (F) PCC7002:pAB225 conjugation clone 1; (G) pAB220 plasmid preparation.

Thus, FIG. 7 demonstrates that, following antibiotic selection, both types of plasmids (the recombinant form of the endogenous plasmid and the wild type form of the endogenous plasmid) continued to persist in the cells. The presence of the endogenous pAQ1 plasmid indicates that full segregation was not achieved in the high salt media.

TABLE 6

| Primer | Sequence |
|---|---|
| pAQ1FlankAFnew | 5′-CTTTCTCTTATGCACAGATGGG-3′ |
| pAQ13939R | 5′-TGATGATGATCGGTAGCT-3′ |
| pAQ13475F | 5′-AGCCATGTGTTATACAGTGT-3′ |
| M13F (−20) | 5′-GTAAAACGACGGCCAGT-3′ |

### Example 6

### Low Salt Segregation

Strain PCC7002:AB125 was created by transforming, via natural transformation, a linear piece of DNA (containing Pnb1A7120-PDC-oop-PrbcL6803-ADHdeg-oop and flanking regions A3 and B3) cut from plasmid pAB125. A clone of nonsegregated PCC7002:pAB125 was cultured in MBG-11 supplemented with vB12 and 100 ug/mL spectinomycin liquid medium for approximately 1 week and retained near max ethanologenic production levels of about 0.07% ethanol (v/v) after incubation of the cells starting at an OD₇₅₀ of about 1.0 for 24 h at 37° C. under continuous light of 320 μEm⁻² s⁻¹.

Following initial selection of ethanol production clones PCC7002:pAB125, full segregation of the transformed marine cyanobacterial cells containing the recombinant form of the endogenous plasmid was accomplished using low salt media as follows: PCC7002:pAB125 clones were cultured in liquid MBG-1 media supplemented with vB12 and about 1×MIC of spectinomycin to an OD₇₅₀ of about 1.5-2.0. 1 mL of the culture was washed once in BG-11 (2.500×g, 10 min spin) and streaked for single colonies on low salt media (BG-11+vB12) plates with various spectinomycin concentrations (sp15, 30, 60, 120, 240, 300, 360, and 500 μg/mL). These segregation plates were incubated at room temperature with low light overnight and then at 37° C. with continuous light or with a 12 h/12 h light/dark cycle for approximately 2 weeks. There was abundant growth (e.g., a lawn) on plates containing spectinomycin at 15 to 120 mg/L, while single colonies formed on plates containing 240 and 300 mg/L of spectinomycin. No colonies were found at higher spectinomycin concentrations.

From the 240 mg/L spectinomycin and 300 mg/L spectinomycin segregation plates, 24 and 48 single colonies were picked and restreaked as small patches on fresh 240 mg/L spectinomycin and 300 mg/L spectinomycin plates. After approximately 1 week all these clones grew into healthy patches under continuous light. Cell samples were taken to prepare DNA for PCR tests to determine whether clones contained endogenous pAQ1 plasmid and/or recombinant pAQ1-pAB125 plasmid. The absence of endogenous pAQ1 plasmid would indicate that the clones were fully segregated.

FIG. 8 shows PCR analysis of 24 clones from a segregation plate containing 240 mg/L spectinomycin. Primers used to amplify by PCR were pAQ1FlankAR and pAQ12774F. The PCR reactions using Synechococcus sp. strain PCC 7002 DNA (depicted as WT) generated a 1.9 Kb PCR product. Clearly, 16 out of 24 clones contained the recombinant form of the endogenous plasmid pAQ1 while the wild type pAQ1 was not detectable as indicated by the absence of 1.9 Kb bands. Clones #2, 15 and 21 showed PCR products of two different sizes. Clones #5, 10 and 16 did not show any PCR products, suggesting that they might contain mutations, such as point mutations or truncations at the primer locations.

As depicted in FIG. 8, 16 out of the 24 clones isolated from the sp300 plate (67%) appeared to be fully segregated, as they generated only the PCR product band of the size expected for the recombinant pAQ1-AB125 and not for wild-type pAQ1. Thus, FIG. 8 depicts successful full segregation of high copy number, recombinant form of an endogenous plasmid in marine cyanobacteria using low salt media.

Transformants of Synechococcus sp. strain PCC 7002 transformed with the integrative vector pAB125 were grown in liquid culture and tested for ethanol production in GC vials, as described herein in the section “Assay for Ethanol Production”. A transformant cell that had not undergone segregation process (control) and several fully segregated clones were tested in the same experiment. Several segregated clones did not produce ethanol, likely due to mutation(s) in the ethanologenic cassette. However, many segregated clones were able to make ethanol.

FIG. 9 is a bar graph depicting levels of ethanol production in cultures of Synechococcus sp. strain PCC 7002 transformed with integrative vector pAB125 (pAQ1B3A3:Pnb1A-PDC-Prbc-ADH_{deg}). The control refers to a culture that did not undergo the segregation process. Three clones, 3-8, 3-12 and 3-24 produced ethanol at levels that were about thirty percent higher than the non-segregated control. The clones analyzed in FIG. 9 were isolated from plates containing spectinomycin at 240 mg/L (sp240). Cultures (2 mL) were grown in 10 mL colorless glass GC vials with constant shaking. The cell density (OD₇₅₀) at the start of the incubation was set at about 1.0. The ethanol level percent, expressed in volume of ethanol per volume of solution, was determined after 24 h incubation at 37° C. under continuous illumination at 190 μEs⁻¹ m⁻².

Thus, the completely segregated clones produced high levels of ethanol, and the ethanologenic construct is stably maintained in marine cyanobacteria without the use of antibiotic or any other selection.

### Example 7

### Test of Plasmid Stability

Two different methods were used to detect plasmid loss. One method used a PCR analysis, while the other method used cell plating. For the PCR analysis, fully segregated clones (#4, 11, 18, and 19 from sp300) and non-segregated clones (#1 and 7 from sp300) of PCC7002-AB125 as well as the original transformant culture that had not gone through the segregation process (control, non-segregated) were grown in 15-mL MBG11 supplemented with vB12 with either sp100 or no antibiotic for about 10 days. DNA was extracted and PCR was carried out using the primer set of pAQ1FlankAR/pAQ12774F.

FIG. 10 is an image of an ethidium bromide-stained agarose gel depicting PCR analysis of recombinant and wild-type (WT) plasmids in both segregated (clone numbers 4, 11, 18, and 19) and non-segregated (clone numbers 1, 7) transformed cells. Control refers to the transformant clone that did not undergo the segregation process and therefore contained both the recombinant and wild-type forms of the pAQ1 plasmids. The cells were grown either without antibiotic (“sp0”) or with 100 mg/L spectinomycin (“sp100”). The positions of the WT and recombinant band are indicated at the right. The expected PCR products were 7 kb for the recombinant plasmid and 1.9 kb for the wild-type (WT) pAQ1 plasmid. DNA extraction buffer (TE beads) and total DNA isolated from wild type PCC 7002 were used as negative and positive PCR controls, respectively. Fully segregated clones 4, 11, 18, and 19 were confirmed to contain only recombinant plasmid and the plasmids were maintained even in the absence of antibiotic. Conversely, as compared to the culture with antibiotic, the absence of antibiotic in the medium led to a stronger PCR product band of the WT plasmid for clone 7.

Moreover, the PCR product band of the recombinant plasmid of the non-segregated clone #7 was clearly much weaker in the culture without antibiotic than that with antibiotic. Thus, the PCR analysis, although semi-quantitative, shows the loss of recombinant form of an endogenous plasmid in non-segregated clones when no selection pressure was maintained.

The above-described clones were also evaluated for plasmid stability using a plating assay. The four fully segregated clones and two non-segregated clones of FIG. 10 were grown in liquid MBG11 supplemented with vB12 without antibiotic for approximately 10 days (from starting OD₇₅₀ of about 0.2 to a final OD₇₅₀ of greater than about 1.0). From each culture, about 2,500 cells were plated for single colonies on MBG11+vB12 plate without antibiotic. Plates were incubated at 28° C. (12 h/12 h light cycle) for about 2 weeks for single colonies to grow. For each clone (either fully segregated or non-segregated), 40 single colonies were randomly picked and restreaked as small patches on MBG11+vB12+sp100 plate and incubated for about 10 days at 28° C. (12 h/12 h light cycle). Plasmid loss was scored by the number of the 40 patches that failed to grow up on selection plate. If the cells of a colony on the non-selection plate had lost the recombinant form of an endogenous plasmid they would not be able to grow on the selection plate. Growth on the selection plates would indicate the persistence of the recombinant plasmid.

As would be expected with non-segregated plasmids, two of the colonies from the non-segregated clone #1 and 13 colonies from the non-segregated clone #7 could not grow on the selection plates, indicating the loss of some of the recombinant plasmids during the culture without spectinomycin selection in the medium. On the contrary, all forty colonies from each of four segregated clones formed healthy cell patches on the selection plates.

Therefore, both the PCR analysis and the plating assay demonstrated that after complete segregation, the recombinant form of an endogenous plasmid was stably maintained in the cell in the absence of antibiotic selection, while non-segregated cells quickly lost the recombinant form of the endogenous plasmid if no selection for the recombinant form of the endogenous plasmid (i.e., the appropriate antibiotic) was present in the medium.

In a separate experiment, plating and PCR tests confirmed that the pAQ1-based integration vector pAB125 in the liquid culture of a fully segregated cell line persisted, was stably maintained, in the absence of antibiotic over six transfers (3-4 weeks between each transfer) for a period of over 5 months. Even at the end of this long duration there was no sign of plasmid loss.

### Example 8

### Full Segregation of a Recombinant pAQ1-Based Shuttle Vector in Low Salt Media

The low salt segregation method for marine cyanobacteria developed for pAQ1-based integrative vector pAB125 was used for full segregation of the pAQ1-based shuttle vector pAB232. Synechococcus sp. strain PCC 7002 was transformed with the pAQ1-based shuttle vector pAB232 according to example 3. Colonies were generated on BG-11 plates containing 240 mg/L spectinomycin. Cells were streaked on plates for regrowth and DNA isolation. Two primer sets were used in the PCR analysis, pAQ1FlankAFnew/pAQ13939R and pAQ13475F/M13F(−20), see Table 6.

FIG. 11 is an image of two ethidium bromide-stained gels demonstrating PCR analysis following segregation of pAQ1-based shuttle vector pAB232 in a low salt media. (A.) PCR using primers set pAQ1FlankAFnew/pAQ13939R and a short extension time of 40 seconds, generating a PCR product of 1.2 kB for the WT plasmid. No PCR product could be generated for the recombinant form of the endogenous plasmid under this condition. (B.) PCR using primer set pAQ13475F/M13F(−20), showing a PCR product of 1.4 Kb for the recombinant plasmid. (C) PCR using primers set pAQ1FlankAFnew/pAQ13939R and a long extension time of 5 min, generating a PCR product of 1.2 kB for the WT plasmid and a PCR product of 7 kB for the recombinant form of the endogenous plasmid. PCR reactions 1 through 24 are reaction containing DNA from different clones of PCC7002:pAB232. PCR reactions 25 through 28 are control reactions with pAB232 plasmid DNA. PCC7002 WT DNA, TE/Bead extraction method control (no DNA control), and DNAse-free water (no DNA control), respectively. Column M is DNA size markers.

pAQ1FlankAFnew/pAQ13939R with a 40 second extension yields a 1.2 kb product for the WT plasmid but no product for the recombinant plasmid. pAQ1 FlankAFnew/pAQ13939R with a 5 minute extension yields 1.2 kb product for WT and 7 kb product for the recombinant plasmid. pAQ13475F/M13F(−20) with a 40 second extension yields no product for the WT, but a 1.4 kb product for the recombinant plasmid.

As depicted in FIG. 11, all clones of PCC7002:pAB232 formed a PCR product expected for the recombinant form of the endogenous plasmid. However, some clones also had the PCR product of the WT plasmid. Clones 1, 2, 6, 7, 8, 11, 14, 16, 17, 22 and 24 showed only the PCR product of the recombinant form of the endogenous plasmid, but no product corresponding to the WT plasmid, indicating that these clones were fully segregated. Thus, 11/24 (46%) of the tested clones were found to be fully segregated when the low salt segregation method was used, as compared to none when the segregation was attempted with high salt medium, as in Example 5. In addition, it was observed that samples 20, 21, and 23 did not form a product with the pAQ13475F/M13F(−20) primer set, but did result in recombinant product with the pAQ1FlankAFnew/pAQ13939R primer set, which would suggest mutations in the pAQ13475F and/or M13F(−20) primer sequences.

### Example 9

### Use of Various Promoters

The use of various promoters to drive expression of the recombinant ethanologenic genes, as well as expression of the resulting proteins and ethanol production, was examined. The ethanologenic expression cassette PcpcB7002-PDC-ADH, for example, was cloned into several of pAQ1-based plasmids from marine cyanobacteria to investigate their potential to carry the recombinant ethanol pathway genes. Several promoters were cloned to control the expression of the ethanol pathway genes, including, for example, pstS(7002), mrgA(ABCC171), rbcL(6803), rbcL(7002), dpsA1(7002), smtB-PsmtA(7002), nb1A(7120), and nb1A(7120). Synechococcus sp. strain PCC7002 was transformed with different constructs, as shown below in Table 7, either as a shuttle vector or linearized DNA fragment containing an ethanologenic gene cassette. Transformant cell lines were grown and tested for ethanol production. Results are shown in Table 7.

TABLE 7

| | | | | | EtOH |
|---|---|---|---|---|---|
| Plasmid | | | Type of | Plasmid | production level |
| Designation* | Expression cassette | Plasmid origin | vector | segregation | (v/v)** |
| pAB338 | PpstS(7002)-Pdc-adh | RSF1010 | shuttle | N/A | 0.023% |
| pAB356 | PmrgA(171)-pdc- | RSF1010 | shuttle | N/A | 0.075% |
| | PrbcL(6803)-adh | | | | |
| pAB218 | PrbcL(7002)-pdc-adh | pAQ1/pUC | shuttle | no | 0.060% |
| pAB220 | PdpsA1(7002)-pdc-adh | pAQ1/pUC | shuttle | no | 0.027% |
| pAB232 | PnblA(7120)-pdc- | pAQ1/pUC | shuttle | no | 0.039% |
| | PrbcL(6803)-adh | | | | |
| AB124 | PrbcL(7002)-pdc- | pAQ1 | integration | no | 0.028% |
| | PrbcL(6803)-adh | | | | |
| AB125 | PnblA(7120)-pdc- | pAQ1 | integration | yes | 0.089% |
| | PrbcL(6803)-adh | | | | |
| AB126 | PdpsA1(7002)-pdc- | pAQ1 | integration | yes | 0.096% |
| | PrbcL(6803)-adh | | | | |

*Prefix “p” refers to plasmid. The absence of the prefix “p” indicates that a DNA fragment, rather than the whole plasmid, is prepared by PCR or restriction digestion of the plasmid and transformed into cyanobacteria cells.
**Ethanol production level was determined using a GC vial assay: cultures (2 mL) were grown in 10 mL colorless glass GC vials with a stir bar for mixing. The ethanol level was determined after incubation for 24 h at 37° C. under continuous illumination at 320 uE/s/m². The cell density (OD₇₅₀) at the start of the incubation was about 1.0. Ethanol produced is expressed as percent vol/vol.

### Example 10

### Use of Fully Segregated pAQ1-Based Recombinant Integrative Vectors to Produce Ethanol in Synechococcus sp. Strain PCC 7002 Cells

In a prophetic example, Synechococcus sp. strain PCC 7002 host cells are transformed with the integrative pAQ1-based plasmids as described above. The transformed cells are treated using the low salt method as above in order to create fully segregated recombinant cells. The cells are tested to confirm that while the recombinant plasmid is present, the original endogenous pAQ1 plasmid is absent. The cells are then scaled up and grown in an outdoor large-scale photobioreactor of about 500 L. The cells are capable of maintaining the recombinant form of an endogenous plasmid for at least several months even without the presence of antibiotics, and ethanol is produced at relatively high levels due to the stability of the recombinant plasmids. The ethanol so produced is collected, distilled, and used for biofuel.

### Example 12

### Use of Fully Segregated pAQ1-Based Recombinant Shuttle Vectors to Produce Compounds of Interest in Cyanobacterial Host Cells

In another prophetic example, pAQ1-based shuttle vectors are prepared that have an antibiotic resistance gene and a gene cassette for a biosynthetic pathway to produce a compound of interest in cyanobacteria. Synechococcus sp. strain PCC 7002 host cells are transformed with these shuttle-type pAQ1-based plasmids, using methods described above. The transformed host cells are then treated using the low salt method as described above in order to create fully segregated recombinant cells. The host cells are tested to confirm that while the recombinant plasmid is present, the original endogenous pAQ1 plasmid is absent. The host cells are also tested to confirm the production of the compound of interest. The cells are then scaled up and grown in an outdoor large-scale photobioreactor of about 500 L with continuous mixing and adequate CO₂ addition. Cell samples are taken every other day to determine the stability of the recombinant plasmid in this culture system.

By use of this method, the cyanobacterial cells would be shown to maintain a high copy number of recombinant plasmids over the several week test period, even without the presence of an antibiotic to select for cells having the recombinant plasmids. The production of the compound of interest over the several week test period would also confirmed. Thus, the suitability of the transformed, fully segregated cyanobacterial host cells for large-scale production of a compound of interest would be demonstrated.

### Example 13

### Assay for Ethanol Production

For characterization of transformed recombinant cyanobacteria cells containing an ethanologenic biosynthetic pathway expression cassette, ethanol production was determined as follows: Cyanobacteria cells are inoculated into fresh medium in shake flasks or bottles. Depending on the promoters used for driving the expression of PDC and ADH genes, appropriate inducer or induction medium are used to induce ethanol production. For determination of ethanol concentration of a liquid culture, samples are removed from the culture for analysis at selected times pre- and post-induction (e.g., 0, 1, 2, 3, 4, 5, 8, 10, 12, 15, 20, or 25 days, etc.) and placed in sealed GC vials. Ethanol and acetaldehyde in the headspace is determined using gas chromatography (GC).

When cultures are incubated in shaking flasks, air exchange is important for cells to grow but it causes loss of volatile products such as ethanol and acetaldehyde. To have a more accurate estimation of the amount of ethanol produced in a culture, growing cells are incubated in sealed GC vials for ethanol synthesis and subsequent GC analysis. Typically, a 2 mL cell culture is pipetted into a 10 mL GC vial. A 1 M sodium bicarbonate solution is added to obtain a final concentration of 20 mM. After capping the vial, 2 mL of 100% CO₂ gas is injected through the cap using a syringe. Immediately, the cap is tilted very slightly to release air pressure inside, and the vial is quickly sealed. A similar procedure can be followed to prepare ethanol and acetaldehyde standards except that no bicarbonate and CO₂ are added. Vials containing cultures or standards are incubated in the light for 24 hours. Cultures are mixed continuously with a magnetic bar in the GC vial on a mixing platform, or the GC vials are placed on a rotary shaker. Following the incubation, ethanol in the headspace is determined as above.

### Example 14

### Full Segregation of a Recombinant pAQ3-Based Vector

Attempts to achieve full segregation of a pAQ3 based vector having antibiotic resistance markers under control of a strong promoter, PpsbA from Amaranthus hybridus, for example, in medium A using the methods provided for in Xu et al. were unsuccessful. But, full segregation of a recombinant pAQ3 vector containing a pdc and an adh gene cassette with different antibiotic resistance markers Tn903 or aadA, both under control of their native promoters, was successful, see FIG. 12 (depicting plasmid TK161(pGEM-AQ3::smtB-PsmtA7002-PDC-D-PrbcL6803 synADH_deg.-Nm (Tn903)) and FIG. 13 (depicting plasmid TK162(pGEM-AQ3::smtB-PsmA₇₀₀₂-PDC-PDrbcL₆₈₀₃-synADH_deg.-Sp/Sm).

To achieve homologous recombination of a pdc/adh/selection marker cassette into endogenous pAQ3, flanking regions were amplified according to the methods described by Xu et al., and above. Transformation of Synechococcus strain PCC 7002 was performed as described by Xu et al. Briefly, about 1 μg of a recombinant precursor plasmid of TK161 and TK162 that had an antibiotic marker under the control of a strong promoter, for example PpsbA from Amaranthus hybridus, was cut with NsiI/SpeI and ApaI/NsiI, respectively, and purified by a column. The linearized DNA was mixed with 2 mL of a well-grown culture of Synechococcus strain PCC 7002 (OD₇₅₀ of 1.5) and incubated overnight with gentle bubbling of air supplemented with 0.5% CO₂. Alternatively, the DNA was mixed with a concentrated culture and incubated for about 16 hours without shaking or bubbling.

After incubating for about 16 hours, the cells were streaked on medium A plates containing 120 μg/mL kanamycin. After one week, colonies appeared and were picked on fresh plates.

Further proceeding with the method of Xu et al, clones containing the ethanologenic cassettes were transferred weekly to medium A plates with a stepwise increase of concentration of the appropriate antibiotic in order to obtain full segregation (150, 250, 400, 600, 800, 1000 μg/mL for kanamycin).

Even after using such high amounts of antibiotics, full segregation was not achieved for the precursor plasmid of TK161 and TK162 having an antibiotic marker under the control of a strong promoter.

Full segregation of the pAQ3 based TK161 and TK162 was obtained by changing the promoter associated with the antibiotic marker from a stronger promoter to a weaker promoter. The kanamycin resistance cassette under the control of the strong promoter PpsbA from Amaranthus hybridus in the precursor plasmid of TK161 and TK162 was exchanged with Tn903 for TK161 and likewise with aadA for TK162. This provided a decrease in the relative amount of enzymes encoded by antibiotic resistance genes Tn903 for kanamycin resistance, and aadA for streptomycin resistance. Thus, without being bound by theory, the effective amount of the antibiotic on the medium A plates was increased to an effective level to allow for the full segregation of pAQ3 based TK161 and TK162 on medium A plates which have about 18 g/L of NaCl.

The method to achieve the full segregation of the pAQ3 based TK161 and TK162 further used higher concentrations of antibiotics when the incubated, transformed cells were plated on medium A plates. Instead of the stepwise increase in concentration as used in Xu et al., the plating started with kanamycin at 450 μg/mL, for TK161, then selected colonies were directly transferred to medium A plates having kanamycin at 1500 μg/mL and then finally transferred to medium A plates having 2000 μg/mL kanamycin, at which point the isolated colonies showed full segregation of the recombinant pAQ3 TK161 plasmid and the endogenous pAQ3 plasmid.

TK162 containing cells were plated on medium A plates with streptomycin at 75 μg/mL and then plated on medium A plates having 300 μg/mL streptomycin and then finally transferred to medium A plates having 500 μg/mL streptomycin.

For PCR verification of putative transformants, kanamycin or streptomycin resistant colonies were transferred from the 2000 μg/mL kanamycin or 500 μg/mL streptomycin medium A plates to new medium A plates with the same concentration of selective antibiotic and grown for 3-7 days. Cells of isolated clones were suspended in 30 μL TE buffer and partially lysed by freezing and thawing. The cell suspension was then centrifuged briefly and kept on ice or at −20° C. As a control, lysate of non-transformed wild-type Synechococcus strain PCC 7002 cells was prepared similarly.

PCR analysis of the isolated plasmid DNA was as follows. A typical PCR mixture contained 1.5 μL of cell lysate, 1 μM of each primer pAQ3.9 (TACGGTGACACCCAACTGAA) and pAQ3.10 (CCCAAAGATTGGGAAAATCA) and 10 μL of DreamTaq Master Mix (2×) (Fermentas) in a total volume of 20 μL.

The complete segregation of pAQ3 based recombinant vectors TK161 and TK162 from endogenous pAQ3 plasmids in several clones isolated according to the methods above and PCR analysis of plasmid DNA isolated therefrom is shown in FIG. 14 and FIG. 15, respectively.

FIG. 14 depicts PCR product bands of TK161 transformants. Eight clones were analyzed by PCR. PCR products were run on an agarose gel and stained with ethidium bromide. Lanes 1-8 depict a single PCR product band correlating to the recombinant pAQ3-based TK161 plasmid, and do not contain endogenous pAQ3 plasmid. Lane 9 contains an endogenous pAQ3 plasmid as a control and lane M is a lane containing size markers.

FIG. 15 depicts PCR product bands of TK162 transformants. Twenty clones were analyzed by PCR. PCR products were then analyzed on an agarose gel and stained with ethidium bromide. Most of the colonies isolated exhibit full segregation of the recombinant pAQ3-based TK162 plasmid from the endogenous pAQ3 plasmid. Lane 21 contains the PCR amplification product of the wild-type pAQ3 plasmid as a control. Lane M is a lane containing size markers.

The production of ethanol in the fully segregated clones containing TK161 and TK162 was ≧0.02% (v/v)/OD×d and ˜0.02% (v/v)/OD×d, respectively depicted in FIG. 16 and FIG. 17, as measured by gas chromatography.

### Example 15

### Additional Embodiment of Full Segregation of a Recombinant pAQ1-Based Vector

Attempts to achieve full segregation of a pAQ1 based vector in medium A using the methods provided for in Xu et al. were unsuccessful. However, full segregation was obtained using a similar strategy as employed for achieving full segregation as presented above for pAQ3 based vectors TK161 and TK162. The antibiotic resistance cassette under the control of the PpsbA promoter was changed to antibiotic resistance cassettes Tn903 or aadA having their native promoters.

For integration into pAQ1, flanking regions were chosen for the generation of an alternative integration site which resulted in a deletion of 57 bp (compared to the flanking regions used in Xu et al. that created a deletion of 1185 bp). Two regions of homology, flanking region pAQ1-FA2 (SEQ ID NO:3) (depicted in FIG. 18) incorporating NsiI/SalI endonuclease restriction sites and flanking region pAQ1-FB2 (SEQ ID NO:4) (depicted in FIG. 19) incorporating NotI/SpeI endonuclease restriction sites were designed to integrate into pAQ1. The flanking regions were amplified from pAQ1 by PCR. The flanking regions pAQ1-FA2 and pAQ1-FB2 were then cloned into a cloning vector (pGEM-TK) via the restriction endonuclease sites NsiI/SalI or NotI/SpeI, respectively.

The cloning vector described above was used as precursor for TK165 (pGEM-AQ1-2::smtB-PsmtA₇₀₀₂-PDC-PrbcL₆₈₀₃-synADH_deg.-Nm (Tn903)) and TK166 (pGEM-AQ1-2::smtB-PsmtA₇₀₀₂-PDC-PrbcL₆₈₀₃-synADH_deg.-Sp) as depicted in FIG. 20 and FIG. 21, respectively. Between the flanking regions (pAQ1-FA2 and pAQ1-FB2), additional heterologous genes, pdc, adh and a kanamycin resistance marker under control of the strong promoter PpsbA was cloned. This precursor plasmid of TK165 and TK166 didn't lead to segregated clones. TK165 and TK166 were generated by replacing PpsbA and the kanamycin resistance marker with Tn903 or aadA having their native promoters.

The complete segregation of recombinant Synechococcus strain PCC 7002 transformed with pAQ1 based recombinant vectors TK165 and TK166 from endogenous pAQ1 plasmids was demonstrated in several clones isolated according to the methods above.

For PCR verification of putative Synechococcus strain PCC 7002 transformants, PCR analysis of the isolated plasmid DNA was as follows. A typical PCR mixture contained 1.5 μL of cell lysate, 1 μM of each appropriate primer pAQ1.14 (GTTACAGCGTGACCAAGCAA) and pAQ1.15 (ATTGGGGTTTTCAGGCTTTT) and 10 μL of DreamTaq Master Mix (2×) (Fermentas) in a total volume of 20 μL.

FIG. 22 depicts PCR products generated from isolated DNA from isolated pAQ1 based recombinant plasmids from two Synechococcus strain PCC 7002 clones transformed with TK165. The PCR products were run on an agarose gel and stained with ethidium bromide. Lanes 1-2 depict a single band correlating to the size of the expected product of a recombinant pAQ1-based TK165 plasmid, and do not contain a band correlating to the size of the expected product of an endogenous pAQ1 plasmid. Lane 3 contains the PCR product obtained from the PCR with endogenous pAQ1. Lane M is a lane containing size markers.

Using the same PCR conditions as above, FIG. 23 depicts the PCR products obtained from isolated DNA from sixteen Synechococcus strain PCC 7002 clones transformed with TK166 as isolated using the methods above. As can be seen in FIG. 23, most of the colonies isolated exhibit a significant segregation of the recombinant pAQ1-based TK166 plasmid from the endogenous pAQ1 plasmid. The isolated clone corresponding to lane 10 demonstrated full segregation of TK166 from endogenous pAQ1 plasmids. Lane 17 contains the PCR product band resulting from the PCR with endogenous pAQ1. Lane M is a lane containing size markers.

The production of ethanol in the fully segregated clones containing TK165 (depicted in FIG. 24) or TK166 (depicted in FIG. 25) was about 0.012% (v/v)/OD×d as measured by gas chromatography.

### OTHER EMBODIMENTS

A number of embodiments of the disclosure have been described. Nevertheless, it will be understood that various modifications may be made without departing from the spirit and scope of the disclosure. Such modifications will become apparent to those skilled in the art from a consideration of this disclosure or practice of the disclosure disclosed herein. Consequently, it is not intended that this disclosure be limited to the specific embodiments disclosed herein, but that it cover all modifications and alternatives coming within the true scope and spirit of the disclosure as embodied in the attached claims.

1. A method for isolating recombinant marine cyanobacteria containing a recombinant form of a high copy number endogenous plasmid and lacking the wild-type form of said high copy number endogenous plasmid, said recombinant form of said high copy number endogenous plasmid comprising a wild-type high copy number endogenous plasmid backbone and an exogenous polynucleotide sequence insert, said exogenous polynucleotide sequence insert comprising a selective marker, said method comprising:
a) culturing a recombinant marine cyanobacteria comprising said recombinant form of said high copy number endogenous plasmid on low salt media comprising concentrations of a selective agent,
b) analyzing plasmid DNA from colonies cultured on a medium of step a) for the presence of said recombinant form of said high copy number endogenous plasmid and for the absence of said wild-type high copy number endogenous plasmid, and
c) isolating recombinant marine cyanobacteria from step b) that contain copies of said recombinant form of said high copy number endogenous plasmid and do not contain said wild-type high copy number endogenous plasmid.

2. The method of claim 1, wherein said low salt media of step a) comprises said selective agent that is an antibiotic at a concentration of from about 1× to about 2000× the minimum inhibitory concentration of said antibiotic.

3. The method of claim 1, wherein said low salt media of step a) comprises said selective agent that is an antibiotic at a concentration of from about 15 μg/mL to about 3000 μg/mL, said antibiotic is selected from the group consisting of streptomycin, spectinomycin, kanamycin, gentamycin, erythromycin, neomycin, rifampin, ampicillin, and zeomycin.

4. The method of claim 1, wherein said exogenous polynucleotide sequence comprises an expression cassette.

5. The method of claim 4, wherein said expression cassette comprises an ethanologenic biosynthetic pathway expression cassette comprising genes encoding pyruvate decarboxylase and alcohol dehydrogenase enzymes.

6. The method of claim 4 wherein said selective marker is selected from the group consisting of Tn903 and aadA, and wherein step a) further comprises the addition of salt to said low salt media to produce a concentration of salt of about 18 g/L.

7. The method of claim 6, wherein said media of step a) comprises said selective agent that is an antibiotic at a concentration of from about 1× to about 2000× the minimum inhibitory concentration of said antibiotic.

8. The method of claim 6, wherein said media of step a) comprises said selective agent that is an antibiotic at a concentration of from about 15 μg/mL to about 3000 μg/mL, said antibiotic is selected from the group consisting of streptomycin, spectinomycin, kanamycin, gentamycin, erythromycin, neomycin, rifampin, ampicillin, and zeomycin.

9. The method of claim 1, wherein said isolated recombinant marine cyanobacteria is selected from the group consisting of Prochlorococcus, Synechocystis, Synechococcus, Chroococcales, Cyanobium, Oscillatoriales, Cyanobacterium, Pleurocapsales, Geitlerinema, Phormidium, Euhalothece, Anabaena, Lyngbya, Spirulina, Nostoc, Pleurocapsa, and Leptolyngbya.

10. The method of claim 1, wherein said isolated recombinant marine cyanobacteria is of the species Synechococcus sp. strain PCC 7002.

11. The method of claim 1, wherein said recombinant marine cyanobacteria of step a) comprises said recombinant form of said high copy number endogenous plasmid comprising a wild-type high copy number endogenous plasmid backbone and an exogenous polynucleotide sequence wherein said recombinant form of said high copy number endogenous plasmid is produced by transforming marine cyanobacteria with said exogenous polynucleotide comprising a selective marker, a pyruvate decarboxylase gene, an alcohol dehydrogenase gene, and flanking regions wherein said flanking regions allow for homologous recombination of said exogenous polynucleotide sequence insert into said wild-type form of said high copy number endogenous plasmid to form said recombinant form of said high copy number endogenous plasmid.

12. The method of claim 11, wherein said low salt media of step a) comprises said selective agent that is an antibiotic at a concentration of from about 1× to about 2000× the minimum inhibitory concentration of said antibiotic.

13. The method of claim 11, wherein said low salt media of step a) comprises said selective agent that is an antibiotic at a concentration of from about 15 μg/mL to about 3000 μg/mL, said antibiotic is selected from the group consisting of streptomycin, spectinomycin, kanamycin, gentamycin, erythromycin, neomycin, rifampin, ampicillin, and zeomycin.

14. The method of claim 11, wherein said exogenous polynucleotide sequence comprises an expression cassette.

15. The method of claim 14, wherein said expression cassette comprises an ethanologenic biosynthetic pathway expression cassette comprising genes encoding pyruvate decarboxylase and alcohol dehydrogenase enzymes.

16. The method of claim 15, wherein said selective marker is selected from the group consisting of Tn903 and aadA, and wherein step a) further comprises the addition of salt to said low salt media to produce a concentration of salt of about 18 g/L.

17. The method of claim 16, wherein said media of step a) comprises said selective agent that is an antibiotic at a concentration of from about 1× to about 2000× the minimum inhibitory concentration of said antibiotic.

18. The method of claim 16, wherein said media of step a) comprises said selective agent that is an antibiotic at a concentration of from about 15 μg/mL to about 3000 μg/mL, said antibiotic is selected from the group consisting of streptomycin, spectinomycin, kanamycin, gentamycin, erythromycin, neomycin, rifampin, ampicillin, and zeomycin.

19. A method for isolating recombinant Synechococcus sp. strain PCC 7002 containing a recombinant form of a high copy number endogenous plasmid and lacking the wild-type form of said high copy number endogenous plasmid, said recombinant form of a high copy number endogenous plasmid comprising a wild-type high copy number endogenous plasmid backbone and an exogenous polynucleotide sequence insert, said exogenous polynucleotide sequence insert comprising a selective marker, said method comprising:
a) transforming Synechococcus sp. strain PCC 7002 comprising said wild-type high copy number endogenous plasmid with an exogenous polynucleotide,
b) culturing said transformed Synechococcus sp. strain PCC 7002 in media comprising a selective agent,
c) culturing said cultured transformed Synechococcus sp. strain PCC 7002 from step b) on low salt media comprising concentrations of a selective agent,
d) selecting individual colonies from a medium of step c),
e) culturing said selected individual colonies from step d) on said medium of step d),
f) selecting colonies from said medium of step e),
g) culturing said selected colonies from step f) on a high salt medium,
h) analyzing plasmid DNA from said high salt medium cultures from step g) for the presence of said recombinant form of a high copy number endogenous plasmid and for the absence of said wild-type high copy number endogenous plasmid,
i) isolating recombinant Synechococcus sp. strain PCC 7002 from step h) that contain copies of said recombinant form of a high copy number endogenous plasmid and do not contain said wild-type high copy number endogenous plasmid, and
j) culturing said isolated recombinant Synechococcus sp. strain PCC 7002 from step i) in a high salt medium that does not contain said selective agent for a period of time.

20. The method of claim 19, wherein the period of time of step j) is less than about 5 months.

21. The method of claim 19, wherein said low salt media of step c) comprises said selective agent that is an antibiotic at a concentration of from about 1× to about 2000× the minimum inhibitory concentration of said antibiotic.

22. The method of claim 19, wherein said low salt media of step a) comprises said selective agent that is an antibiotic at a concentration of from about 15 μg/mL to about 3000 μg/mL, said antibiotic is selected from the group consisting of streptomycin, spectinomycin, kanamycin, gentamycin, erythromycin, neomycin, rifampin, ampicillin, and zeomycin.

23. The method of claim 19, wherein said exogenous polynucleotide of step a) is a plasmid comprising an expression cassette.

24. The method of claim 23, wherein said expression cassette comprises an ethanologenic biosynthetic pathway expression cassette comprising genes encoding pyruvate decarboxylase and alcohol dehydrogenase enzymes.

25. The method of claim 24 wherein said selective marker is selected from the group consisting of Tn903 and aadA, and wherein step c) further comprises the addition of salt to said low salt media to produce a concentration of salt of about 18 g/L.

26. The method of claim 25, wherein said media of step c) comprises said selective agent that is an antibiotic at a concentration of from about 1× to about 2000× the minimum inhibitory concentration of said antibiotic.

27. The method of claim 25, wherein said media of step c) comprises said selective agent that is an antibiotic at a concentration of from about 15 μg/mL to about 3000 μg/mL, said antibiotic is selected from the group consisting of streptomycin, spectinomycin, kanamycin, gentamycin, erythromycin, neomycin, rifampin, ampicillin, and zeomycin.

28. The method of claim 19, wherein said exogenous polynucleotide of step a) comprises an exogenous polynucleotide comprising a selective marker, a pyruvate decarboxylase gene, an alcohol dehydrogenase gene, and flanking regions wherein said flanking regions allow for homologous recombination of said exogenous polynucleotide sequence insert into said wild-type form of said high copy number endogenous plasmid to form said recombinant form of said high copy number endogenous plasmid.

29. The method of claim 28, wherein said low salt media of step c) comprises said selective agent that is an antibiotic at a concentration of from about 1× to about 2000× the minimum inhibitory concentration of said antibiotic.

30. The method of claim 28, wherein said low salt media of step c) comprises said selective agent that is an antibiotic at a concentration of from about 15 μg/mL to about 3000 μg/mL, said antibiotic is selected from the group consisting of streptomycin, spectinomycin, kanamycin, gentamycin, erythromycin, neomycin, rifampin, ampicillin, and zeomycin.

31. The method of claim 28 wherein said selective marker is selected from the group consisting of Tn903 and aadA, and wherein step c) further comprises the addition of salt to said low salt media to produce a concentration of salt of about 18 g/L.

32. The method of claim 31, wherein said media of step c) comprises said selective agent that is an antibiotic at a concentration of from about 1× to about 2000× the minimum inhibitory concentration of said antibiotic.

33. The method of claim 31, wherein said media of step c) comprises said selective agent that is an antibiotic at a concentration of from about 15 μg/mL to about 3000 μg/mL, said antibiotic is selected from the group consisting of streptomycin, spectinomycin, kanamycin, gentamycin, erythromycin, neomycin, rifampin, ampicillin, and zeomycin.

34. A genetically-modified Synechococcus sp. strain PCC 7002 host cell comprising a recombinant form of a high copy number endogenous plasmid, wherein no corresponding high copy number endogenous plasmid is present in said host cell.

35. The host cell of claim 34, wherein said recombinant form of said high copy number plasmid comprises a gene encoding pyruvate decarboxylase and a gene encoding alcohol dehydrogenase.

36. The host cell of claim 35, wherein said host cell produces ethanol.


SEQUENCE LISTING (4 sequences)

SEQ ID NO: 1 - DNA, 685 bp, Synechococcus PCC7002
ggtagcccctagactgtgccagatcataagcctcactgagggtgagggcactaccgggggcatgagctcgcccaagagattcagcgaccggggcgatcgcccttggtaattctctcaggcgctcaacttttttaggcgctgatcgagctcttggatttgctgttggatgtcagcaagctctggaatttcccgattctctgatgggagatccaaaaattctcgcagtccctcaatcacgatatcggtcttggatcgccctgtagcttccgacaactgctcaattttttcgagcatctctaccgggcatcggaatgaaattaacggtgttttagccatgtgttatacagtgtttacaacttgactaacaaatacctgctagtgtatacatattgtattgcaatgtatacgctattttcactgctgtctttaatggggattatcgcaagcaagtaaaaaagcctgaaaaccccaataggtaagggattccgagcttactcgataattatcacctttgagcgcccctaggaggaggcgaaaagctatgtctgacaaggggtttgacccctgaagtcgttgcgcgagcattaaggtctgcggatagcccataacatacttttgttgaacttgtgcgcttttatcaaccccttaagggcttgggagcgttttatacgagtgcggggaaatc

SEQ ID NO: 2 - DNA, 626 bp, Synechococcus PCC7002
gatccctgagcttgtcgagaaagatgatgatgggtggtactctcagctccagctcgaatactacttaaccgttgggaaagcattctgctctgcccgcgaccgggcgaaatatgaccagctccaacatgagggctttgtatttaagccggatatcaacaggcgatcgctctcaccaaagattcacctgttagagctactcaacatccatcagttcttaaaaccaggggtgacattcaccggggcgagccttgaagggttcaaggaaaattgtttgcggtatgccaagccgatcaagtggattcttggcagaacgatcaccgacaaaatgagcccgctcgaaattgctcaggcgctcctaggcaagcttgaccggaaattggaatacaaggggcgctttggatcgcgggataaccgtcagcggtctatgaggcgatcgcccctaacgatcagcgcgaaaaggtctttgctcattggttacagcgtgaccaagcaaaattaggggccgtgtccaacccctgtataaatagatttattcaggaggcttagacccgtgatcgaaatactcgttgtgcagctctcccttggcaatcccaaacaatctcaagatttgctctgcggtatcggga

SEQ ID NO: 3 - DNA, 683 bp, Synechococcus PCC7002
atatgcatctccaacatgagggctttgtatttaagccggatatcaacaggcgatcgctctcaccaaagattcacctgttagagctactcaacatccatcagttcttaaaaccaggggtcattcaccggggcgagccttgaagggttcaaggaaaattgtttgcggtatgccaagccgatcaagtggattcttggcagaacgatcaccgacaaaatgagcccgctcgaaattgctcaggcgctcctaggcaagcttgaccggaaattggaatacaaggggcgctttggatcgcgggataaccgtcagcgggtctatgaggcgatcgcccctaacgatcagcgcgaaaaggtctttgctcattggttacagcgtgaccaagcaaaattaggggccgtgtccaacccctgtataaatagatttattcaggaggcttagacccgtgatcgaaatactcgttgtgcagctctcccttggcaatcccaaacaatctcaagatttgctctgcggtatcgggacgttttatgcccttgcggaaagcgcctttgctcttctggtagcccctagactgtgccagatcataagcctcactgagggtgagggcactaccgggggcatgagctcgcccaagagattcagcgaccggggcgatcgcccttggtaattctctcaggcgctgtcgacat

SEQ ID NO: 4 - DNA, 519 bp, Synechococcus PCC7002
tagcggccgctggaatttcccgattctctgatgggagatccaaaaattctcgcagtccctcaatcacgatatcggtcttggatcgccctgtagcttccgacaactgctcaattttttcgagcatctctaccgggcatcggaatgaaattaacggtgttttagccatgtgttatacagtgtttacaacttgactaacaaatacctgctagtgtatacatattgtattgcaatgtatacgctattttcactgctgtctttaatggggattatcgcaagcaagtaaaaaagcctgaaaaccccaataggtaagggattccgagcttactcgataattatcacctttgagcgcccctaggaggaggcgaaaagctatgtctgacaaggggtttgacccctgaagtcgttgcgcgagcattaaggtctgcggatagcccataacatacttttgttgaacttgtgcgcttttatcaaccccttaagggcttgggagcgttttatacgagtgcggggaactagtta